(19) United States
(12) Patent Application Publication
(10) Pub. No.: US 2010/0174181 A1
(43) Pub. Date: Jul. 8, 2010

(54) LIQUID INJECTOR, FLUOROSCOPIC IMAGING SYSTEM, AND COMPUTER PROGRAM

(75) Inventor: Shigeru Nemoto, Tokyo (JP)

Correspondence Address: TUROCY & WATSON, LLP, 127 Public Square, 57th Floor, Key Tower, CLEVELAND, OH, 44114, US

(73) Assignee: NEMOTO KYORINDO CO., LTD., Tokyo (JP)

(21) Appl. No.: 12/601,574

(22) PCT Filed: May 29, 2008
(86) PCT No.: PCT/JP2008/001337

§ 371 Date: Nov. 24, 2009

(30) Foreign Application Priority Data
May 30, 2007 (JP) 2007-143333

(51) Int. Cl. A61B 6/00; G06Q 10/00; G06Q 50/00

(52) U.S. Cl. 600/431; 705/2; 600/476

(57) ABSTRACT

Once a patient ID and injection control data are input to a liquid injector and liquid injection is executed, the injection control data and injection history data are registered with the patient ID in a PACS. When the same patient is to undergo the second or subsequent liquid injection, the previous injection control data and injection history data are acquired by inputting the patient ID, and set as renewed injection control data. Such arrangement eliminates the need to input the same injection control data for the patient. Further, since the injection control data of each patient is registered and acquired utilizing the patient ID as index, erroneous setting of inappropriate injection control data can be automatically prevented, when the patient undergoes the injection. The liquid injector allows, therefore, easily setting the injection control data, and yet prevents liquid injection based on inappropriate injection control data.

### TECHNICAL FIELD

The present invention relates to a liquid injector that injects a medical liquid to a patient from whom fluoroscopic image data is to be taken, a fluoroscopic imaging system incorporated with the liquid injector, and a computer program for the liquid injector.

### BACKGROUND ART

Imaging diagnostic apparatuses currently available for picking up a tomographic image, which is fluoroscopic image data of a patient, include a Computed Tomography (CT) scanner, a Magnetic Resonance Imaging (MRI) equipment, a Positron Emission Tomography (PET) equipment, and an ultrasonic diagnostic equipment. Also, medical equipments that pick up a vascular image, which is another fluoroscopic image data of the patient, include a CT angiographic equipment, a Magnetic Resonance Angiographic (MRA) equipment, and so forth.

When one of such equipments is used, the patient often undergoes an injection of a medical liquid, also called a medical fluid, or simply liquid as the case may be, such as a contrast medium or physiological saline, and liquid injectors that automatically execute the injection are currently in practical use. A popular liquid injector retains a liquid syringe loaded with the liquid, and a piston member is press-inserted into the cylinder member of the syringe to thereby inject the liquid into the patient's body.

Although the imaging diagnostic apparatus can work on a stand-alone basis, normally a fluoroscopic imaging system is constituted, including the imaging diagnostic apparatus as part thereof. Such fluoroscopic imaging system includes, for example, a chart management unit, an imaging management unit, a imaging diagnostic apparatus, a data storage unit, and an image viewer.

The chart management unit is generally called a Hospital Information System (HIS), and is utilized to manage so-called electronic medical records. The electronic medical records each correspond to a patient.

For example, when a patient is to undergo a fluoroscopic image data pickup, the chart management unit makes up imaging order data based on the patient's electronic medical record. The imaging order data is generated with respect to each imaging job of picking up the fluoroscopic image data of the patient.

More specifically, the imaging order data includes, for example, imaging job ID (identity) representing exclusive identification data, identification data of the imaging diagnostic apparatus, the patient ID, and date and time of the start and finish of the image pickup.

Such imaging order data is provided to the imaging management unit from the chart management unit. The imaging management unit is generally called a Radiology Information System (hereinafter, RIS), and serves to store the imaging order data used for picking up a fluoroscopic image data of the patient.

The imaging diagnostic apparatus acquires the imaging order data from the imaging management unit, and executes the imaging job. In other words, the imaging diagnostic apparatus picks up the fluoroscopic image data of the patient according to the imaging order data. The fluoroscopic image data is allocated with at least a part of the imaging order data in the imaging diagnostic apparatus, and then output to the data storage unit.

The data storage unit, generally called a Picture Archive and Communication System (PACS) or alike, stores therein the fluoroscopic image data allocated with the imaging order data.

To the data storage unit, an image viewer, generally called a viewer, is connected. The image viewer reads out the fluoroscopic image data utilizing, for example, the imaging order data as the retrieval key, and displays that fluoroscopic image data.

It is to be noted that the imaging management unit is usually engaged in managing a plurality of imaging order data. Accordingly, one of the plurality of imaging order data managed by the imaging management unit has to be selectively provided to the imaging diagnostic apparatus. For this purpose, the imaging management unit is designed either as a push-type or as a pull-type.

The push-type imaging management unit selects one of the plurality of imaging order data under the management, for example through manual operation by the operator. The push-type imaging management unit transmits, upon receipt of an acquisition request for the imaging order data from the imaging diagnostic apparatus, the selected one of the imaging order data, in response thereto.

To the pull-type imaging management unit, the imaging diagnostic apparatus transmits an order retrieval key with the acquisition request for the imaging order data. The order retrieval key is composed of an imaging job ID for example, of the imaging order data.

Then the imaging management unit retrieves the imaging order data with the order retrieval key, and transmits the imaging order data thus retrieved as response to the imaging diagnostic apparatus. Upon receipt of the legitimate imaging order data, the imaging diagnostic apparatus picks up the fluoroscopic image data of the patient in correspondence with the imaging order data.

On the other hand, in the case where a plurality of imaging order data is retrieved and returned, the imaging diagnostic apparatus selects one of the plurality of imaging order data received, through manual operation by the operator for example.

In addition, once the imaging order data transmitted by the imaging management unit is fixed in the imaging diagnostic apparatus as above, such effect is notified to the imaging management unit. Accordingly, the pull-type imaging management unit can also identify the specific imaging order data used for picking up the fluoroscopic image data by the imaging diagnostic apparatus.

Regarding the foregoing fluoroscopic imaging system, various proposals have been made (for example, patented documents 1 and 2).

[Patented document 1] JP-A No. 2001-101320

[Patented document 2] JP-A No. 2005-198808

### DISCLOSURE OF THE INVENTION

In the foregoing fluoroscopic imaging system, the liquid injector injects liquid such as a contrast medium to the patient, and the imaging diagnostic apparatus picks up fluoroscopic image data of the patient according to the imaging order data.

However, the contrast medium has to be injected to the patient at an appropriate speed and in an appropriate quantity. Accordingly, the operator has to decide the injection rate and the quantity to be injected based on the region to be imaged, body weight of the patient and so on, and input such conditions to the liquid injector as injection control data. Such task is, however, so complicated that it is difficult for an unskilled operator to properly perform.

Besides, with the conventional liquid injector it is possible to execute the liquid injection only if the injection control data is available, even though the injection control data actually does not properly correspond to the patient who is to undergo the fluoroscopic image data pickup. Therefore, injection to the patient based on inappropriate injection control data cannot be prevented.

The present invention has been accomplished in view of the foregoing problem, with an object to provide a fluoroscopic imaging system that allows easily setting the injection control data that is appropriate for the patient, in the liquid injector.

According to the present invention, there is provided a first liquid injector for use in a fluoroscopic imaging system including an imaging diagnostic apparatus that picks up fluoroscopic image data of a patient, and the liquid injector that injects a medical liquid to the patient from whom the fluoroscopic image data is to be picked up, comprising a data input unit that acquires a patient ID of an individual patient; a liquid injection mechanism that executes injection of the medical liquid; an injection control unit that controls an action of a liquid injection mechanism based on injection control data; a data registration unit that registers the injection control data utilized for controlling the action together with the patient ID; a data acquisition unit that acquires the injection, control data corresponding to the acquired patient ID, from the data registration unit; and a control setting unit that sets the acquired injection control data in the injection control unit.

With the liquid injector according to the present invention, once the patient ID and the injection control data are input and the liquid injection is executed, the injection control data is registered together with the patient ID. Then, when the same patient is to undergo the second or subsequent liquid injection, the previous injection control data is acquired based on the patient ID input, and is set.

According to the present invention, there is provided a second liquid injector for use in a fluoroscopic imaging system including an imaging diagnostic apparatus that picks up fluoroscopic image data of a patient, and the liquid injector that injects a medical liquid to the patient from whom the fluoroscopic image data is to be picked up, comprising a data input unit that acquires a patient ID of an individual patient; a liquid injection mechanism that executes injection of the medical liquid; an injection control unit that controls an action of a liquid injection mechanism based on injection control data; a history generation unit that generates injection history data including an action history of the liquid injection mechanism corresponding to the injection control data; a data registration unit that registers the generated injection history data together with the patient ID; a data acquisition unit that acquires the injection history data corresponding to the acquired patient ID, from the data registration unit; and a control setting unit that sets the acquired injection history data as the injection control data, in the injection control unit.

With the liquid injector according to the present invention, once the patient ID and the injection control data are input and the liquid injection is executed, the injection history data is generated and registered together with the patient ID. Then, when the same patient is to undergo the second or subsequent liquid injection, the previous injection history data is acquired based on the patient ID input, and is set as renewed injection control data.

According to the present invention, there is provided a third liquid injector for use in a fluoroscopic imaging system including an imaging diagnostic apparatus that picks up fluoroscopic image data of a patient, the liquid injector that injects a medical liquid to the patient from whom the fluoroscopic image data is to be picked up, and a data storage unit that stores the fluoroscopic image data together with a patient ID of an individual patient, comprising a data input unit that acquires the patient ID; a liquid injection mechanism that executes injection of the medical liquid; an injection control unit that controls an action of a liquid injection mechanism based on injection control data; a data registration unit that registers the injection control data utilized for controlling the action in the data storage unit in association with the fluoroscopic image data; a data acquisition unit that acquires the injection control data corresponding to the acquired patient ID, from the data storage unit; and a control setting unit that sets the acquired injection control data in the injection control unit.

With the liquid injector according to the present invention, once the patient ID and the injection control data are input and the liquid injection is executed, the injection control data is registered together with the patient ID. Then, when the same patient is to undergo the second or subsequent liquid injection, the injection control data is acquired from the data storage unit based on the patient ID input, and is set.

According to the present invention, there is provided a fourth liquid injector for use in a fluoroscopic imaging system including an imaging diagnostic apparatus that picks up fluoroscopic image data of a patient, the liquid injector that injects a medical liquid to the patient from whom the fluoroscopic image data is to be picked up, and a data storage unit that stores the fluoroscopic image data together with a patient ID of an individual patient, comprising a data input unit that acquires the patient ID; a liquid injection mechanism that executes injection of the medical liquid; an injection control unit that controls an action of a liquid injection mechanism based on injection control data; a history generation unit that generates injection history data including an action history of the liquid injection mechanism corresponding to the injection control data; a data registration unit that registers the generated injection history data in the data storage unit in association with the fluoroscopic image data; a data acquisition unit that acquires the injection history data corresponding to the acquired patient ID, from the data storage unit; and a control setting unit that sets the acquired injection history data as the injection control data, in the injection control unit.

With the liquid injector according to the present invention, once the patient ID and the injection control data are input and the liquid injection is executed, the injection history data is generated and registered together with the patient ID. Then, when the same patient is to undergo the second or subsequent liquid injection, the injection history data is acquired from the data storage unit based on the patient ID input, and is set as renewed injection control data.

According to the present invention, there is provided a first fluoroscopic imaging system including an imaging diagnostic apparatus that picks up fluoroscopic image data of a patient, a liquid injector that injects a medical liquid to the patient from whom the fluoroscopic image data is to be picked up, and a data storage unit that stores the fluoroscopic image data together with a patient ID of an individual patient, comprising the third liquid injector according to the present invention; and the data storage unit that stores injection control data input from the liquid injector, together with the patient ID.

According to the present invention, there is provided a second fluoroscopic imaging system including an imaging diagnostic apparatus that picks up fluoroscopic image data of a patient, a liquid injector that injects a medical liquid to the patient from whom the fluoroscopic image data is to be picked up, a data storage unit that stores the fluoroscopic image data together with a patient ID of an individual patient, comprising the fourth liquid injector according to the present invention; and the data storage unit that stores injection history data input from the liquid injector, together with the patient ID.

According to the present invention, there is provided a first computer program for use with the first liquid injector according to the present invention, comprising causing the liquid injector to execute a data input process including acquiring a patient ID of an individual patient, an injection control process including controlling an action of the liquid injection mechanism based on injection control data, a data registration process including registering the injection control data utilized for controlling the action, together with the patient ID, a data acquisition process including acquiring the injection control data corresponding to the acquired patient ID from the data registration unit, and a control setting process including setting the acquired injection control data in the injection control unit.

According to the present invention, there is provided a second computer program for use with the second liquid injector according to the present invention, comprising causing the liquid injector to execute a data input process including acquiring a patient ID of an individual patient, an injection control process including controlling an action of the liquid injection mechanism based on injection control data, a history generation process including generating injection history data including an action history of the liquid injection mechanism corresponding to the injection control data, a data registration process including registering the generated injection history data, together with the patient ID, a data acquisition process including acquiring the injection history data corresponding to the acquired patient ID input, from the data registration unit, and a control setting process including setting the acquired injection history data in the injection control unit as the injection control data.

According to the present invention, there is provided a third computer program for use with the third liquid injector according to the present invention, comprising causing the liquid injector to execute a data input process including acquiring a patient ID, an injection control process including controlling an action of the liquid injection mechanism based on injection control data, a data registration process including registering the injection control data in the data storage unit in association with the fluoroscopic image data, a data acquisition process including acquiring the injection control data corresponding to the acquired patient ID from the data storage unit, and a control setting process including setting the acquired injection control data in the injection control unit.

According to the present invention, there is provided a fourth computer program for use with the fourth liquid injector according to the present invention, comprising causing the liquid injector to execute a data input process including acquiring a patient ID, an injection control process including controlling an action of the liquid injection mechanism based on injection control data, a history generation process including generating injection history data including an action history of the liquid injection mechanism corresponding to the injection control data, a data registration process including registering the generated injection history data in the data storage unit in association with the fluoroscopic image data, a data acquisition process including acquiring the injection history data corresponding to the acquired patient ID from the data storage unit, and a control setting process including setting the acquired injection history data in the injection control unit as the injection control data.

It is to be noted that each constituent of the present invention has only to be capable of performing its function, and may be constituted in a form of, for example, an exclusive hardware that performs a predetermined function, a data processor in which a predetermined function is incorporated as a computer program, a predetermined function realized in a data processor by a computer program, and an optional combination thereof.

Also, the constituents of the present invention do not necessarily have to be individually independent, but may be configured such that a plurality of constituents constitutes a single member, a constituent is composed of a plurality of members, a constituent is a part of another constituent, a part of a constituent and a part of another constituent overlap, and so forth.

With the liquid injector according to the present invention, once the patient ID and the injection control data are input and the liquid injection is executed, the injection control data and the injection history data are registered together with the patient ID. Then, when the same patient is to undergo the second or subsequent liquid injection, the previous injection control data and the injection history data are acquired based on the patient ID input, and are set as renewed injection control data. Such arrangement eliminates the need to repeat the input of the injection control data with respect to the same patient. Further, since the injection control data of each patient is registered and acquired based on the patient ID, the liquid injection to the patient based on erroneous setting of inappropriate injection control data can be automatically prevented.

### BRIEF DESCRIPTION OF THE DRAWINGS

The above and other objects, features and advantages will become more apparent through a preferred embodiment described hereunder and the following accompanying drawings.

FIG. 1 is a schematic block diagram showing a logical structure of a liquid injector according to an embodiment of the present invention;

FIG. 2 is a schematic block diagram showing a logical structure of a fluoroscopic imaging system;

FIG. 3 is a block diagram showing a physical structure of the fluoroscopic imaging system;

FIG. 4 is a perspective view showing an appearance of a fluoroscopic imaging unit of a CT scanner and an injection head of the liquid injector;

FIG. 5 is a perspective view showing the appearance of the liquid injector;

FIG. 6 is an exploded perspective view showing the injection head of the liquid injector and a liquid syringe;

FIG. 7 is a schematic block diagram showing another logical structure of the liquid injector;

FIG. 8 is a schematic front view showing a screen of the liquid injector, displaying icons of body parts and a condition screen in blank;

FIG. 9 is a schematic front view showing a screen displaying the body part and a region to be imaged that have been selected;

FIG. 10 is a schematic front view showing a screen displaying injection control data;

FIG. 11 is a schematic front view showing a screen displaying a guidance message indicating that acquisition of a patient ID is in process;

FIG. 12 is a schematic front view showing a screen displaying a guidance message indicating an acquisition error of the patient ID;

FIG. 13 is a schematic front view showing a screen displaying an example of the injection condition data;

FIG. 14 is a schematic front view showing a screen displaying another example of the injection condition data;

FIG. 15 is a schematic front view showing a screen displaying liquid condition data;

FIG. 16 is a schematic front view showing a screen indicating the injection control data that has been set;

FIG. 17 is a schematic front view showing a screen displaying a guidance message indicating a reference error of the patient ID;

FIG. 18 is a schematic front view showing a screen displaying a time-based graph representing a liquid injection process based on the injection control data manually set;

FIG. 19 is a schematic front view showing a screen displaying a time-based graph representing a liquid injection process based on the liquid condition data and injection condition data that have been automatically set;

FIG. 20 is a flowchart showing a first half of a process performed by the liquid injector;

FIG. 21 is a flowchart showing a second half of the process performed by the liquid injector;

FIG. 22 is a schematic time chart showing a processing sequence of the fluoroscopic imaging system.

FIG. 23 is a perspective view showing an appearance of an injection head of a modified liquid injector; and

FIG. 24 is a perspective view showing an appearance of an injection head of another modified liquid injector.

### BEST MODE FOR CARRYING OUT THE INVENTION

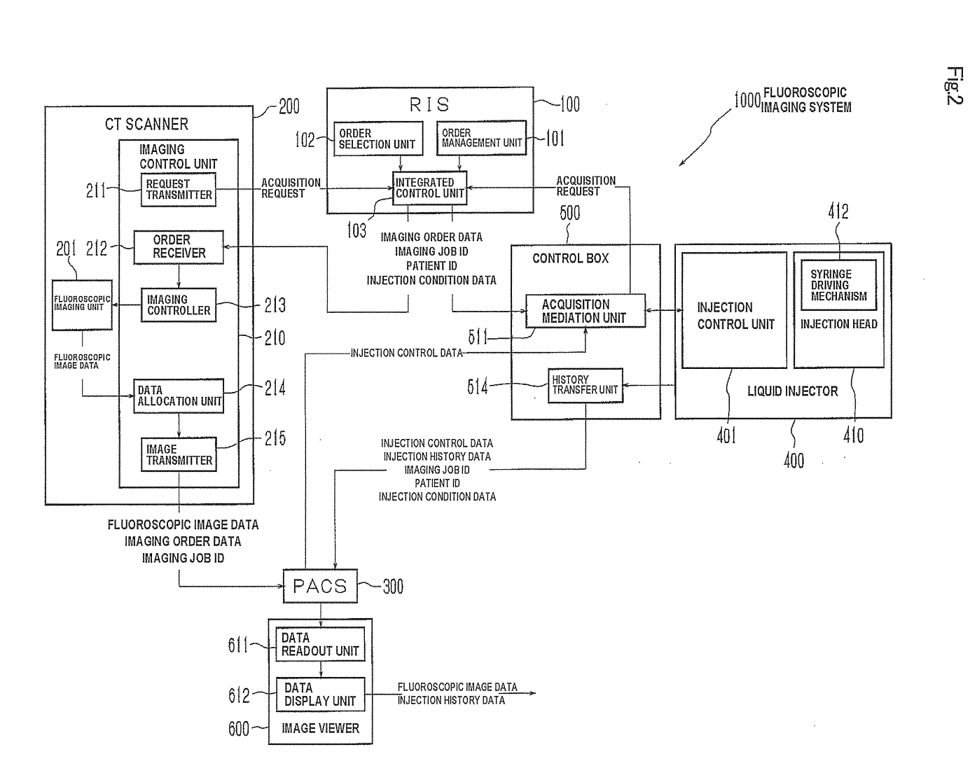

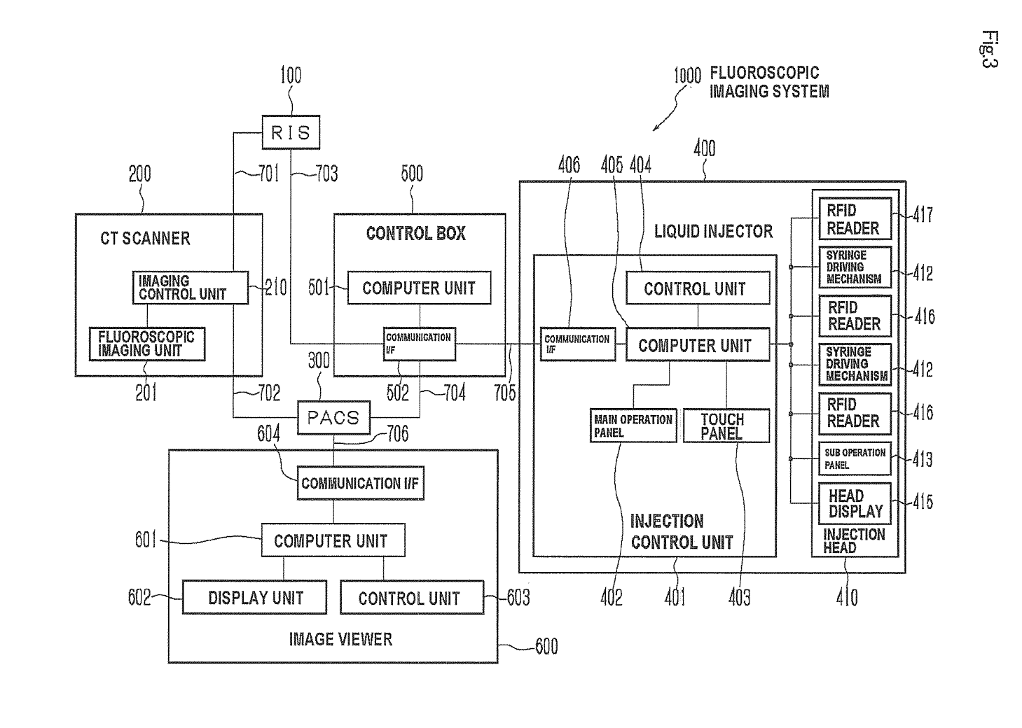

Hereunder, an embodiment of the present invention will be described referring to the drawings. A fluoroscopic imaging system 1000 according to the embodiment of the present invention includes, as shown in FIGS. 2 and 3, a RIS 100 which serves as an imaging management unit, a CT scanner 200 which serves as an imaging diagnostic apparatus, a PACS 300 which serves as a data storage unit, a liquid injector 400, a control box 500 which serves as a data control unit, and an image viewer 600.

In the fluoroscopic imaging system 1000 according to this embodiment, the CT scanner 200 is connected to the RIS 100 and the PACS 300, through communication networks 701, 702 such as a Local Area Network (LAN), as illustrated.

The control box 500 is also connected to the RIS 100, the PACS 300, and the liquid injector 400 through communication networks 703 to 705. To the PACS 300, the image viewer 600 is connected through the communication network 706.

The fluoroscopic imaging system 1000 according to this embodiment is based on what is known as Digital Imaging and Communications in Medicine (DICOM). Accordingly, the respective units 100 to 600 of the fluoroscopic imaging system 1000 mutually communicate according to DICOM specification.

In the fluoroscopic imaging system 1000 according to this embodiment, one each of the CT scanner 200, the PACS 300, the liquid injector 400, and the control box 500 are provided, and all the combinations of these units are on a one-to-one basis.

The RIS 100 according to this embodiment is constituted of a known computer unit, in which an exclusive computer program is installed. In the RIS 100, an order management unit 101, an order selection unit 102, and an integrated control unit 103 are logically realized as the functions thereof, when the computer unit executes the corresponding processes according to the computer program.

The order management unit 101 corresponds to a storage device such as a hard disc drive (HDD), and serves to manage the imaging order data used for picking up fluoroscopic image data of the patient, in other words shooting a fluoroscopic image and thereby generating the fluoroscopic image data of the patient, with the exclusive identification data.

The imaging order data includes text data such as an imaging job ID which is the exclusive identification data, the identification data of the CT scanner 200, date and time of the start and finish of the image pickup, the patient ID of each individual patient, personal data of the patient such as body weight, sex and age, various data on the patient's disease, body part or region to be imaged, and the product name of the contrast medium employed as the medical liquid, also called a medical fluid, or simply liquid as the case may be.

The order selection unit 102 corresponds to a function assigned to the central processing unit (hereinafter, CPU), including executing a predetermined process according to an input through a keyboard, and selects one from a plurality of imaging order data according to the input by the operator.

The integrated control unit 103 corresponds to a function assigned to the CPU including transmitting and receiving various data through a communication interface (I/F), and returns the selected one of the imaging order data according to a acquisition request received from the CT scanner 200 or the control box 500.

The CT scanner 200 according to this embodiment includes, as shown in FIG. 3, a fluoroscopic imaging unit 201 which is the image-pickup execution mechanism, and an imaging control unit 210. The fluoroscopic imaging unit 201 shoots the fluoroscopic image data of the patient. The imaging control unit 210 controls the action of the fluoroscopic imaging unit 201.

To be more detailed, the imaging control unit 210 is constituted of a computer unit, in which an exclusive computer program is installed. In the imaging control unit 210, a request transmitter 211, an order receiver 212, an imaging controller 213, a data allocation unit 214, and an image transmitter 215 are logically realized as the functions thereof, when the computer unit executes the corresponding process according to the computer program.

The request transmitter 211 corresponds to a function assigned to the CPU including transmitting and receiving various data through the communication interface (I/F), and transmits the acquisition request for the imaging order data to the RIS 100. The order receiver 212 receives the imaging order data returned from the RIS 100.

The imaging controller 213 controls the action of the fluoroscopic imaging unit 201 according to the imaging order data received. The data allocation unit 214 allocates the imaging order data to the fluoroscopic image data picked up by the fluoroscopic imaging unit 201.

The image transmitter 215 transmits the fluoroscopic image data allocated with the imaging order data to the PACS 300. Here, the fluoroscopic image data thus generated is composed of, for example, bit map data of the tomographic image.

The PACS 300 according to this embodiment is constituted of a database server, in which also an exclusive computer program is installed. The PACS 300 receives the fluoroscopic image data allocated with the imaging order data from the CT scanner 200, and stores the received data.

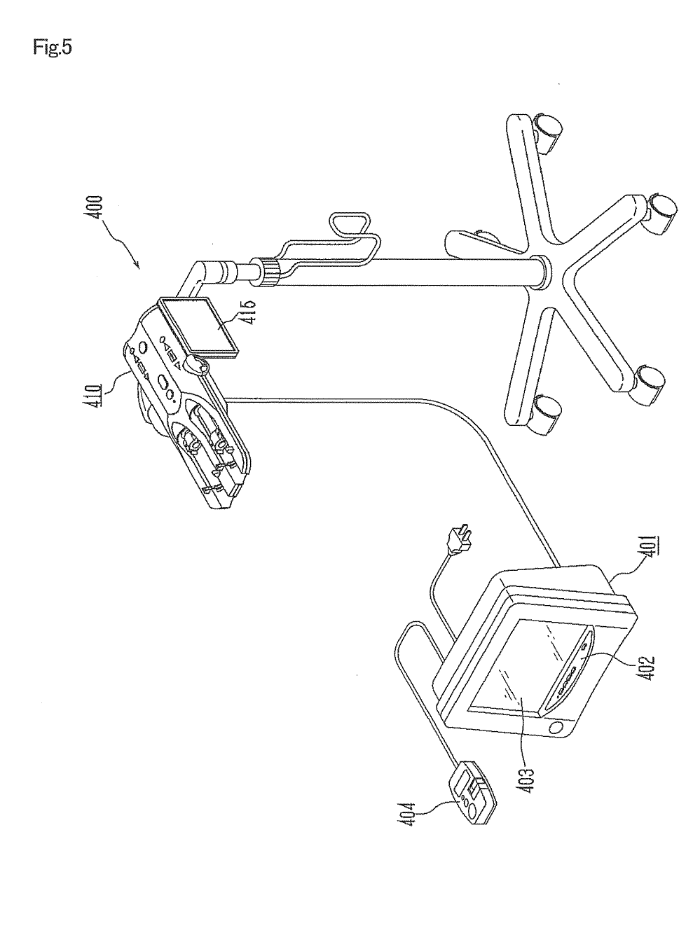

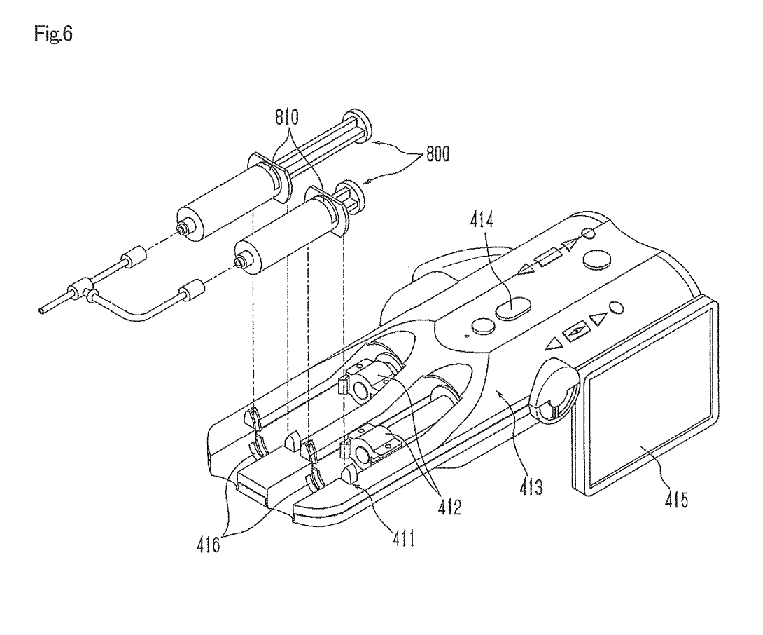

The liquid injector 400 according to this embodiment includes, as shown in FIG. 5, an injection control unit 401 and an injection head 410. The injection control unit 401 controls the action of the injection head 410. The injection head 410 drives a liquid syringe 800, also called a fluid syringe or medical syringe, removably attached thereto as shown in FIG. 6, to thereby inject a liquid into the patient.

To be more detailed, the injection control unit 401 includes, as shown in FIG. 3, a main operation panel 402, a touch panel 403, a controller 404, a computer unit 405, a communication I/F 406.

The injection head 410 includes a syringe holding mechanism 411 that retains the liquid syringe 800, a syringe driving mechanism 412 serving as the liquid injection mechanism that drives the liquid syringe 800, a sub operation unit 413 used to input an action instruction to the syringe driving mechanism 412, a head display 415 serving as the data display device that outputs various data for display, and so forth.

The sub operation unit 413 includes a final confirmation switch 414 which will be subsequently described. The head display 415 is directly fixed to a rear lateral portion of the injection head 410, and located close to the syringe holding mechanism 412 and the syringe driving mechanism 412.

Here, the liquid syringe 800 according to this embodiment is available in various types, some of which include a RFID chip 810 installed at a predetermined position. To the injection head 410, an RFID reader 416 is attached at such a position that enables making wireless communication with an RFID chip 810 only when the liquid syringe 800 is retained by the syringe holding mechanism 412 properly in place.

The RFID chip 810 of the liquid syringe 800 contains at least liquid condition data regarding the liquid, registered therein. To be more detailed, the liquid syringe 800 is of what is known as a prefilled type shipped with the liquid loaded in advance, and hence the liquid condition data is registered in the RFID chip 810 prior to the shipment.

The liquid condition data may include, for example, the data of the loaded liquid such as product name, product ID, chemical classifications, ingredients, viscosity, and expiry date, as well as the data of the liquid syringe 800 such as capacity, pressure resistance, cylinder bore, piston stroke, and lot number.

The product ID of the liquid is registered based on the chemical classifications, ingredients and chemical structure, and is not associated with the syringe capacity and the like. For example, in the case where the products of a company A and a company B are available as heart contrast medium for CT scanning, if the chemical classifications, such as whether water-soluble or oil-based, ionic or anionic, monomer type or dimer type, is different the product IDs become different, though the type of the liquid, “heart contrast medium for CT scanning”, is the same.

Further, although the type of the liquid and chemical classifications are the same, if the ingredients are different the product IDs become different, and even though the type of the liquid, chemical classifications and ingredients are the same, if the chemical structure of even a single ingredient is different, the product IDs become different.

On the other hand, in the case where an identical liquid is loaded in the prefilled liquid syringes of 200 ml and 500 ml in capacity, the product ID of the liquid is the same, though the liquid syringes are different as products by the capacity.

To the computer unit 405 of the liquid injector 400, the respective units cited above are connected. The computer unit 405 integrally controls the computer program, in which the respective units connected to the computer unit 405 are implemented.

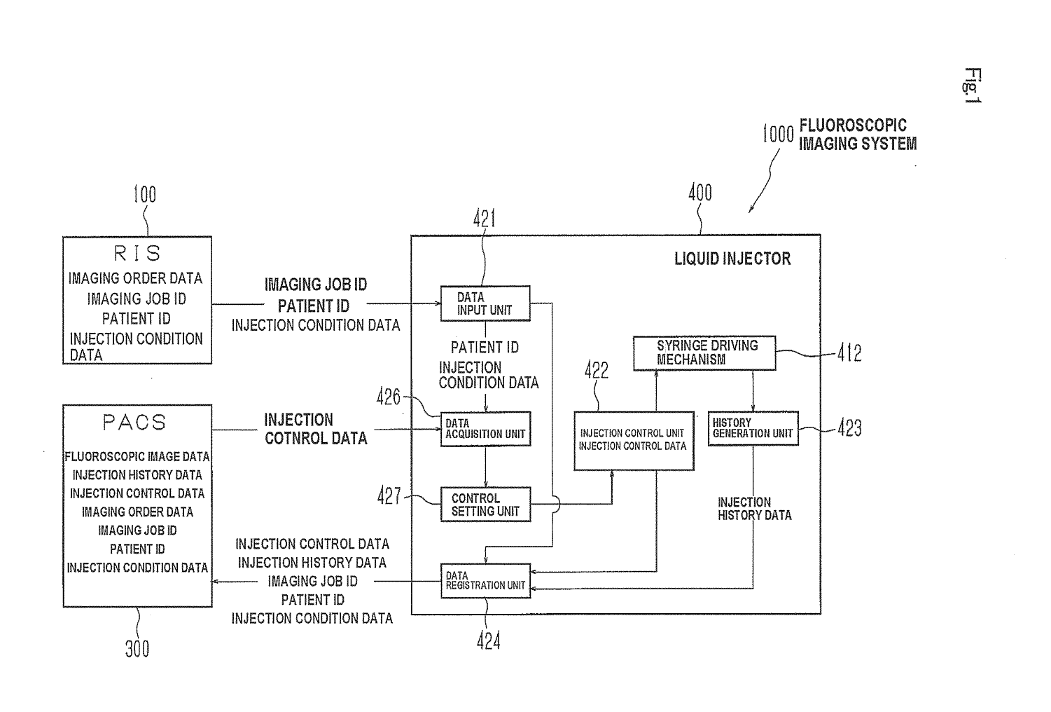

Accordingly, in the liquid injector 400 the following units are logically realized as the functions thereof, as shown in FIG. 1, namely a data input unit 421 that acquires the patient ID, an injection control unit 422 that controls the action of the syringe driving mechanism 412, a data registration unit 424 that registers the injection control data utilized for the action control in the PSCS 300 in association with the fluoroscopic image data, a data acquisition unit 426 that acquires the injection control data corresponding to the acquired patient ID from the PACS 300 through the control box 500, a control setting unit 427 that sets the acquired injection control data in the injection control unit 422.

In other words, the units 421, 424, 426 of the liquid injector 400 correspond to the function of executing data communication with the RIS 100 and the PACS 300 through the communication I/F 406, to be performed by the computer unit 405 according to the foregoing computer program, and the other units 422, 423, 427 correspond to the function of executing various data processings, to be performed by the computer unit 405.

The data input unit 421 accepts an input of an acquisition request for the patient ID, and transmits the acquisition request that has been input to the control box 500. Then once a part of the imaging order data is acquired from the RIS 100 through the control box 500, the patient ID can be acquired as a part of the imaging order data.

The data input unit 421 also receives an input of a predetermined injection condition data related to the liquid injection condition, and the data registration unit 424 registers the injection control data together with the patient ID and the injection condition data.

Then the data acquisition unit 426 acquires the injection control data corresponding to the patient ID and the injection condition data that have been input. The injection condition data includes, for example, the body part and the region to be imaged, and is acquired from the RIS 100 as a part of the imaging order data, together with the patient ID.

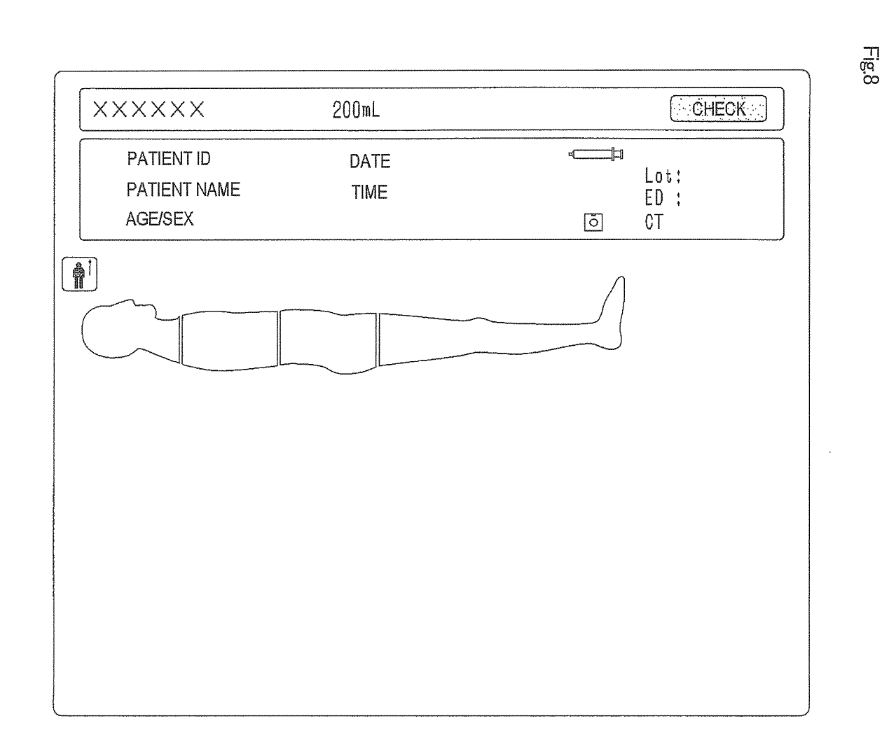

To be more detailed, on the touch panel 403 of the injection control unit 401, an operating icon for inputting the acquisition request, including a profile of a human body and an icon of “i”, is displayed for example in a left upper region of the initial screen for inputting the injection control data, as shown in FIG. 8.

By inputting the acquisition request through manipulating the operating icon, a part of the imaging order data is acquired as the injection condition data, through the control box 500. Then out of the injection condition data, the patient ID and the region to be imaged are set in the injection control unit 422 as at least a part of the injection control data.

The injection control data includes, for example, protocol data in which a moving stroke and pressure of the syringe driving mechanism 412 are specified for different time points, by a predetermined command.

The liquid injector 400 further includes a condition selection unit (not shown) that selects one of the injection control data based on a predetermined condition, out of those acquired in correspondence with the patient ID. Accordingly, the control setting unit 427 sets the selected injection control data in the injection control unit 422. In this case, the condition selection unit selects the latest one out of the plurality of injection control data acquired.

Also, the liquid injector 400 includes a history generation unit 423 that generates injection history data including the action history of the syringe driving mechanism 412 according to the injection control data, and the data registration unit 424 also registers the injection history data in the PACS 300, together with the injection control data.

The injection history data includes image data of a time-based graph in which, for example, one of the horizontal axis and the vertical axis indicates the lapse of time and the other the injection rate, and is allocated with text data such as the injection control data, the patient ID, and the injection job ID and so forth.

The units 421 to 427 are utilized as above for automatically setting the injection control data. However, the liquid injector 400 according to this embodiment can also be manually operated for setting new injection control data and modifying the setting.

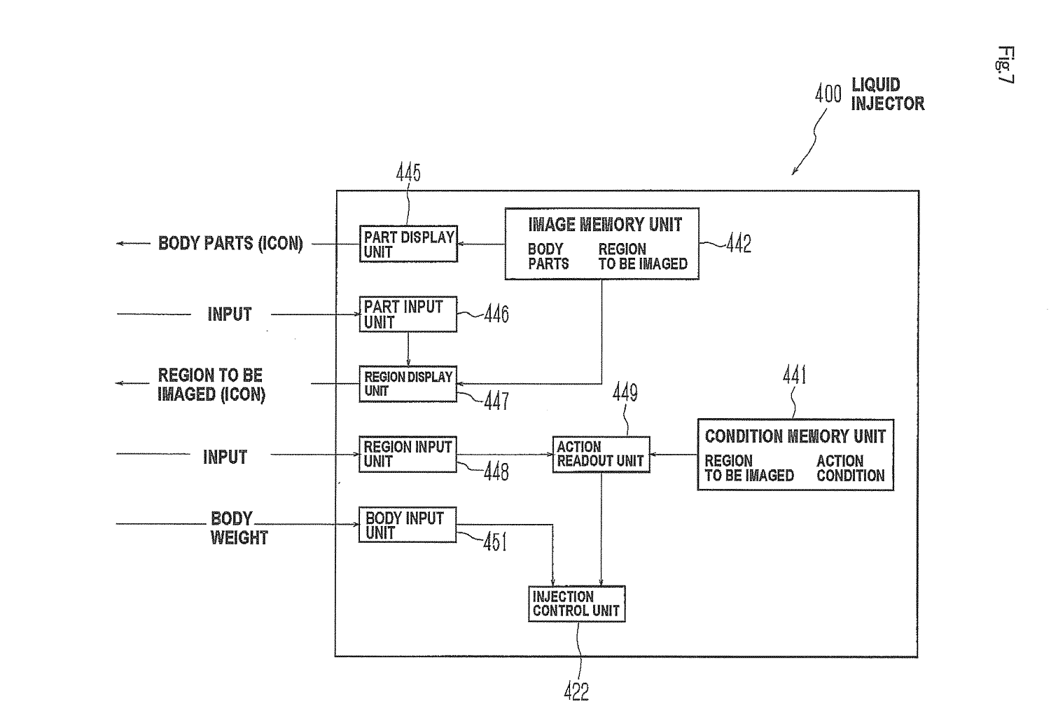

Further, as shown in FIG. 7, in the liquid injector 400 units such as a condition memory unit 441, an image memory unit 442, a part display unit 445, a part input unit 446, a region display unit 447, a region input unit 448, an action readout unit 449, and a body input unit 451 are logically realized as the functions thereof.

The image memory unit 442 of the liquid injector 400 contains the memory of a plurality of human body parts and a multitude of regions to be imaged, in association therebetween. The part display unit 445 displays the icon of the plurality of body parts stored in the image memory unit 442, in a layout corresponding to a human body shape.

The part input unit 446 accepts an input for selecting one of the plurality of body parts displayed on the part display unit 445, as an input of one of the injection condition data. The region display unit 447 outputs and displays an icon of at least a region to be imaged, according to the body part selected through the part input unit 446. The region input unit 448 accepts an input for selecting the region to be imaged displayed on the region display unit 447, as one of the injection condition data.

More specifically, in the liquid injector 400 the plurality of body parts includes the head, chest, abdomen, and leg, and the icons representing each of them are registered in the computer unit 405.

When a predetermined operation is executed with the liquid injector 400, the icons of the head, chest, abdomen, and leg, are displayed in a layout corresponding to a human body shape, in an upper portion of the touch panel 403, as shown in FIG. 8.

Further, icons representing the brain, chin, and neck are registered as a plurality of regions to be imaged, in association with the icon of the head, which is one of the body parts displayed. Likewise, icons of the heart and lung are registered in association with the icon of the chest; icons of the stomach, lever, and so forth in association with the icon of the abdomen; and icons of the upper leg and lower leg in association with the icon of the leg.

Then once one of the icons of the human body shape representing the plurality of body parts displayed on the touch panel 403 is manually touched, an icon representing the scanner mechanism is output and displayed above the selected icon only, and only the manually operated icon is lit up while all the remaining icons are turned off (not shown).

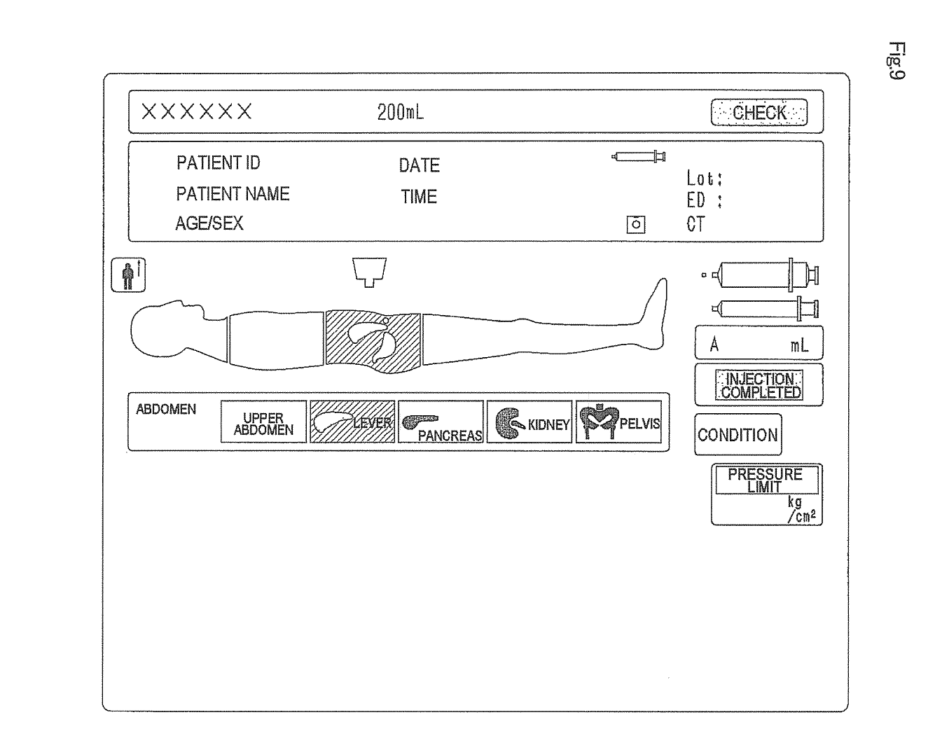

At the same time, below the selected icon, the icons of the plurality of corresponding regions to be imaged are output and displayed. Then when one of the icons representing the plurality of regions to be imaged is manually touched, only the selected icon is lit up and the others are turned out, as shown in FIG. 9.

The condition memory unit 441 stores working condition data of the syringe driving mechanism 412, with respect to each of the multitude of regions to be imaged of the human body. The working condition data is specified, for example, in terms of a total injection amount of a contrast medium to each region to be imaged of the human body.

The action readout unit 449 reads out the working condition data corresponding to the region to be imaged selected through the region input unit 448, from the condition memory unit 441, and sets the data in the injection control unit 422 as a part of the injection control data.

The body input unit 451 accepts an input of body weight, as physical information of the patient who is to undergo the fluoroscopic image data pickup, and sets the body weight in the injection control unit 422 as a part of the injection control data.

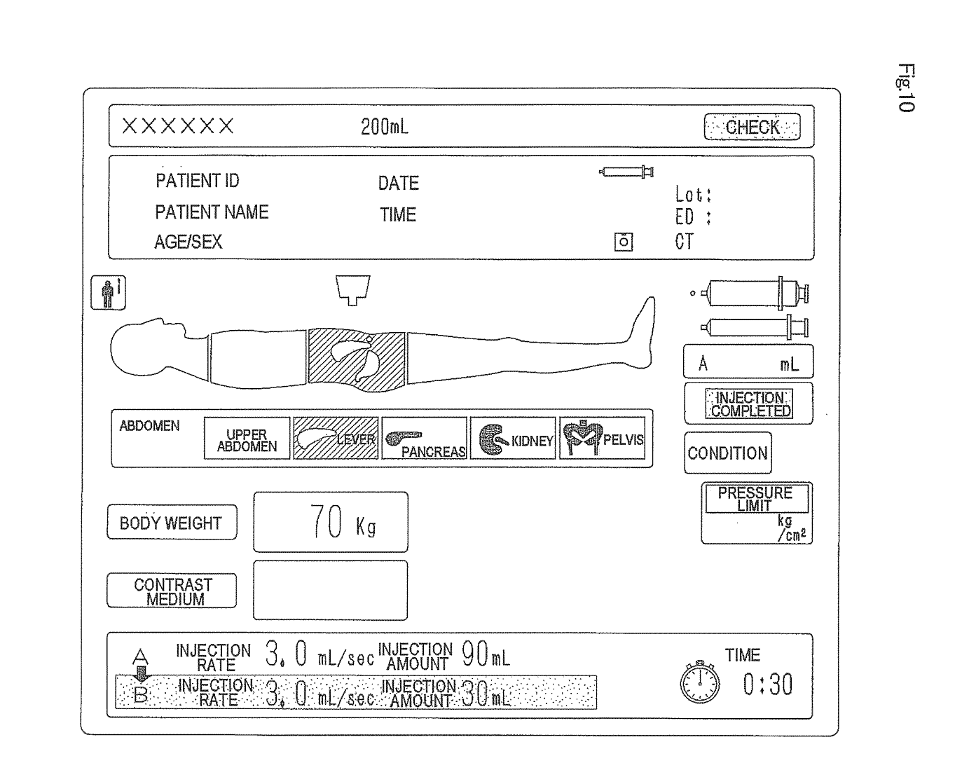

To be more detailed, once the operating icon of “condition” is manually touched after manual selection of the region to be imaged by touching the relevant icon as above, the screen becomes ready to accept an input of body weight, injection amount, injection time and so on. Then upon inputting the value representing the body weight, such value is displayed, and set as a part of the injection control data as shown in FIG. 10.

The control box 500 according to this embodiment includes, as shown in FIG. 3, a computer unit 501 in which an exclusive computer program is installed, and a communication I/F 502.

In the control box 500 also, the computer unit 501 executes various processes according to the computer program. Accordingly, units such as an acquisition mediation unit 511, a history transfer unit 514, and so on are logically realized in the control box 500 as the function thereof.

The acquisition mediation unit 511 acquires the imaging order data from the RIS 100 according to the acquisition request received from the, and returns a part of the acquired imaging order data to the liquid injector 400, as a part of the injection control data. The history transfer unit 514 receives the injection history data from the liquid injector 400, and transfers it to the PACS 300.

Accordingly, the PACS 300 according to this embodiment not only stores the fluoroscopic image data received from the CT scanner 200, but also stores the injection history data received from the control box 500, as described above.

And, as already stated, the fluoroscopic image data is allocated with the imaging order data, and the imaging job ID of that imaging order data is allocated to the injection history data. Accordingly, the imaging order data and the injection history data are stored in the PACS 300, mutually associated via the imaging job ID.

The image viewer 600 according to this embodiment also includes a computer unit in which an exclusive computer program is installed. The image viewer 600 includes, as shown in FIG. 3, a computer unit 601, a display unit 602, a controller 603, a communication I/F 604.

The image viewer 600 includes, as shown in FIG. 2, a data readout unit 611 and a data display unit 612, which are realized when the computer unit 601 executes the corresponding process according to the computer program.

The data readout unit 611 corresponds to a function assigned to the computer unit 601 for making access to the PACS 300 through the communication I/F 604 according to the computer program and the data input to the controller 603, and reads out the fluoroscopic image data and the injection history data associated via the imaging job ID, from the PACS 300.

The data display unit 612 corresponds to the function assigned to the computer unit 601 for causing the display unit 602 to display the data received through the communication I/F 604, and displays the fluoroscopic image data and the injection history data that have been read out.

It is to be noted that the foregoing computer programs of the RIS 100 are described as software for causing the RIS 100 to, for example, store the imaging order data in which the imaging job ID, the patient ID and the injection condition data are specified, select one from a plurality of imaging order data according to an input by an operator, return the selected imaging order data according to the acquisition request from the CT scanner 200 or the control box 500, and so forth.

The computer program of the CT scanner 200 is described as software for causing the imaging control unit 210 to, for example, transmit the acquisition request for the imaging order data to the RIS 100 according to an input by the operator, receive the imaging order data returned from the RIS 100, control the action of the fluoroscopic imaging unit 201 according to the imaging order data that has been received, allocate the fluoroscopic image data picked up by the fluoroscopic imaging unit 201 with the imaging order data, and transmit the fluoroscopic image data allocated with the imaging order data to the PACS 300.

The computer program of the liquid injector 400 is described as software for causing the computer unit 405 to, for example, control the action of the syringe driving mechanism 412 according to the injection control data, generate the injection history data based on the action control, register the generated injection history data and the injection control data utilized for the action control, in the PACS 300 in association with the fluoroscopic image data, acquire the patient ID from the RIS 100 as a part of the imaging order data, acquire the injection control data corresponding to the acquired patient ID from the PACS 300, set the acquired injection control data in the injection control unit 422, and so forth.

The computer program of the control box 500 is described as software for causing the computer unit 501 to, for example, acquire the imaging order data from the RIS 100 according to the acquisition request from the liquid injector 400, return the patient ID etc. of the acquired imaging order data to the liquid injector 400, receive the injection condition data and the injection history data from the liquid injector 400, output the received injection condition data and injection history data to the PACS 300.

The computer program of the PACS 300 is described as software for causing the PACS 300 to, for example, receive the fluoroscopic image data allocated with the imaging order data from the CT scanner 200 and store the fluoroscopic image data, and receive the injection condition data and the injection history data allocated with the imaging job ID of the imaging order data from the control box 500, and store the injection history data.

The computer program of the image viewer 600 is described as software for causing the computer unit 601 to, for example, read out the fluoroscopic image data, the injection condition data and the injection history data mutually associated via the imaging job ID from the PACS 300, and display the fluoroscopic image data, the injection condition data and the injection history data that have been read out.

Hereunder, a procedure of picking up the fluoroscopic image data of the patient with the fluoroscopic imaging system 1000 thus configured according to this embodiment will be sequentially described. To start with, the operator registers in advance the imaging order data in the RIS 100.

The imaging order data is composed of the text data including the imaging job ID, the identification data of the CT scanner 200, date and time of the start and finish of the image pickup, and the region to be imaged. The imaging order data is normally made up based on the electronic medical record of each patient.

Accordingly, the ID, name and body weight of the patient are also registered in the imaging order data. Further, the type and size of the syringe needle used to inject the contrast medium are also registered in this embodiment.

However, the imaging order data includes those data necessary for the CT scanner 200 to execute the imaging job, and the data that enables identifying the injection job of the liquid injector 400 is not contained.

When the imaging job is executed with such imaging order data registered in the RIS 100, the operator may manually operate the RIS 100, to thereby select one of the imaging order data corresponding to the imaging job.

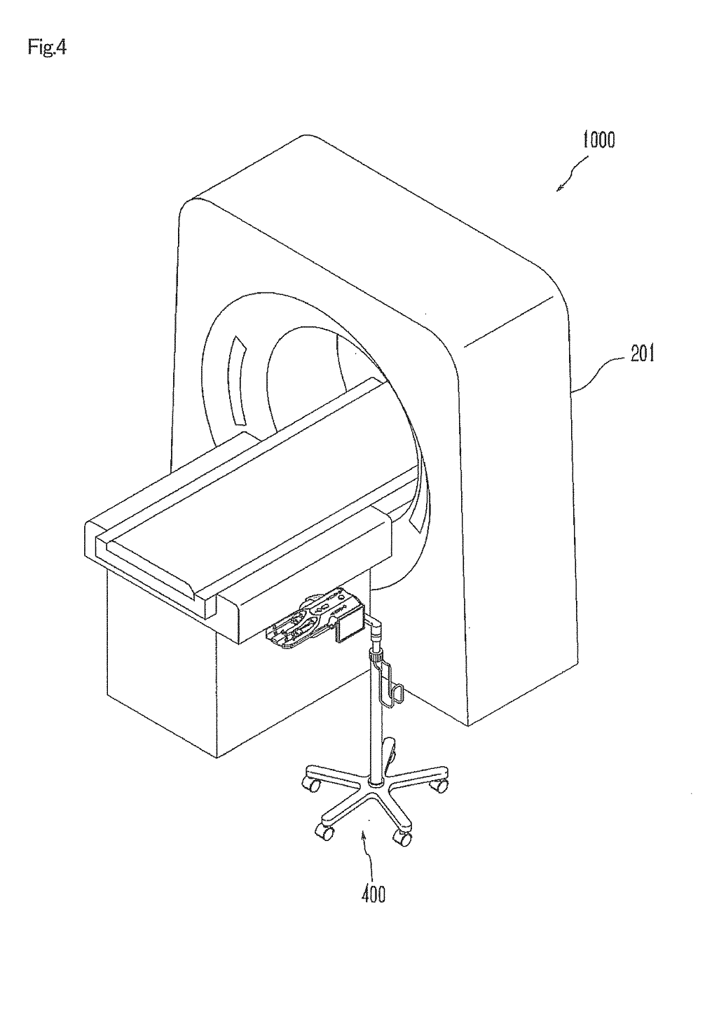

Meanwhile at the actual site of the imaging job, the liquid injector 400 is located close to the fluoroscopic imaging unit 201 of the CT scanner 200, as shown in FIG. 4. Then the liquid syringe 800 is connected to the patient (not shown) in the fluoroscopic imaging unit 201 through an extension tube, and the liquid syringe 800 is loaded onto the injection head 410 of the liquid injector 400.

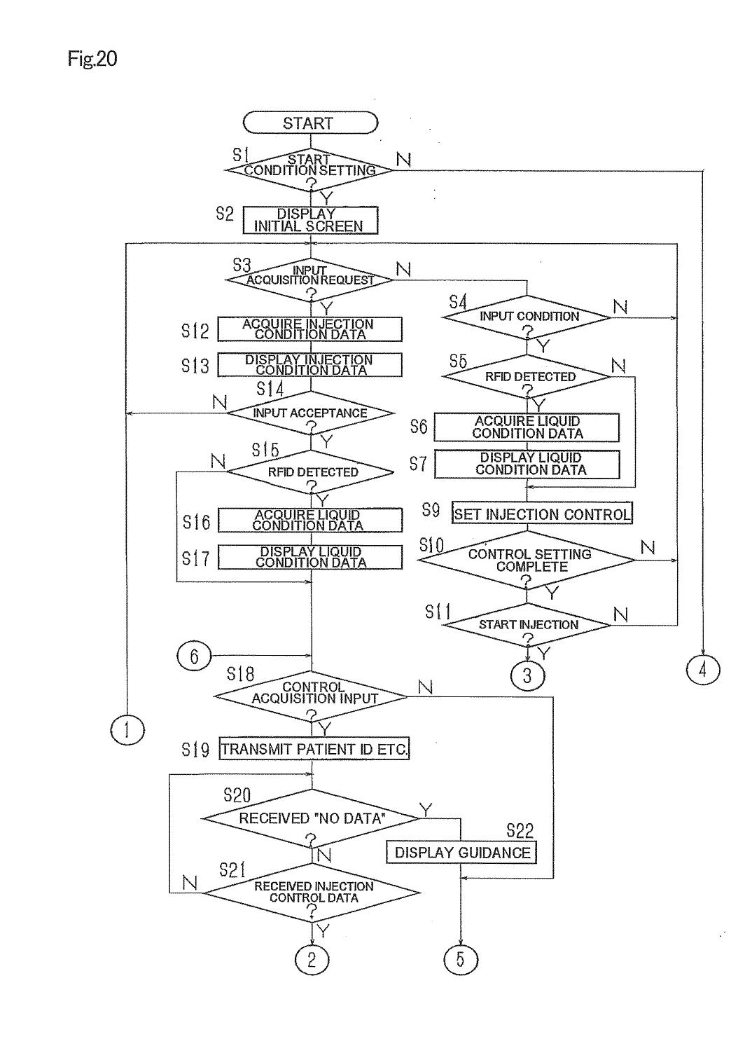

Once the operator activates the liquid injector 400 for example by an inputting action through the main operation panel 402 of the injection control unit 401 as shown in FIG. 20 (step S1), the icons representing a plurality of body parts are displayed on the touch panel 403, as shown in FIG. 8 (step S2).

The liquid injector 400 according to this embodiment does not permit the action control on the syringe driving mechanism 412 based on the injection control data, in the initial stage where the injection control data is not set. While the liquid injector 400 accepts manual setting of the entirety of injection control data at the stage where the initial screen is displayed as above, it is also possible to automatically set a part of the injection control data based on the imaging order data.

In the case of manual setting, the operator presses with a finger one of the plurality of icons representing the body parts displayed on the touch panel 403. Then only the selected part of the icon is lit up while all the remaining parts are turned off, and an icon of the scanner mechanism is displayed above the selected icon of the body part.

At the same time, below the selected part, icons of a plurality of regions to be imaged corresponding to the selected body part are read out and displayed in the selection screen. When the operator inputs one of the icons by a press of a finger, only the selected icon is lit up and the others are turned out, as shown in FIG. 9.

Once the region to be imaged is thus selected, in the liquid injector 400 the action condition data corresponding to the region to be imaged is read out and set as the injection control data. Also, as shown in FIG. 10, the body weight of the patient, injection rate, total injection amount, injection time and so on are input as the injection control data to the main operation panel 402, by the operator (step S4).

At this stage, the liquid injector 400 according to this embodiment also confirms with the RFID reader 416 whether the RFID chip 810 is mounted in the liquid syringe 800 (step S5).

In the case where the RFID chip 810 is mounted in the liquid syringe 800, the RFID reader 416 acquires the liquid condition data (step S6). The liquid condition data includes, as already stated, various data on the loaded liquid such as the product name and expiry, and various data on the liquid syringe 800 such as the capacity and lot number.

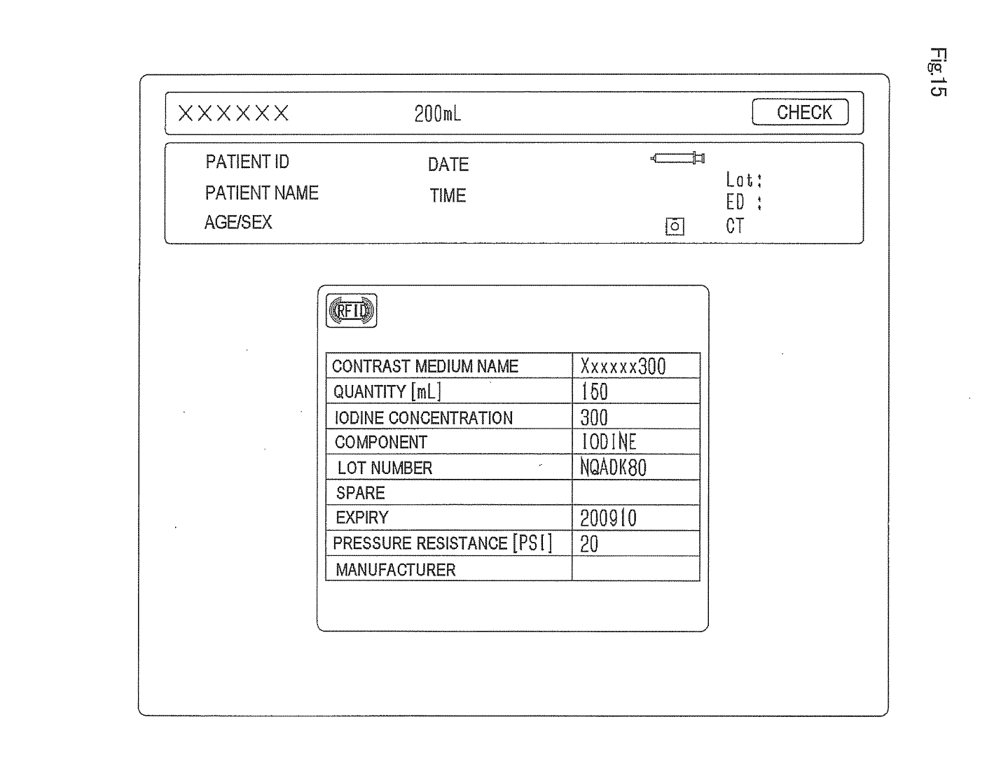

A part of the liquid condition data thus acquired is output for display on the touch panel 403 of the injection control unit 401 and the head display 415 of the injection head 410, as shown in FIG. 15 (step S7).

At this stage, a predetermined “RFID” logo mark appears on the touch panel 403 and the head display 415, indicating that the liquid condition data being displayed has been acquired from the RFID chip 810 in the liquid syringe 800.

Then the operator confirms the liquid condition data displayed as above and inputs the injection control data. Once the setting of the injection control data has been thus completed (steps S9, S10), the liquid injector 400 becomes ready to accept the input of the instruction to start the injection.

Inputting the starting instruction through the touch panel 403 (step S11) activates the syringe driving mechanism 412 to according to the injection control data set as above, so that the contrast medium and physiological saline is properly injected to the patient.

The fluoroscopic imaging system 1000 according to this embodiment also allows, however, automatically setting the injection control data in the liquid injector 400, in addition to the foregoing manual setting. More specifically, the liquid injector 400 according to this embodiment also displays the operating icon of “acquisition request” in an upper left region on the initial screen of the injection job, as shown in FIG. 8.

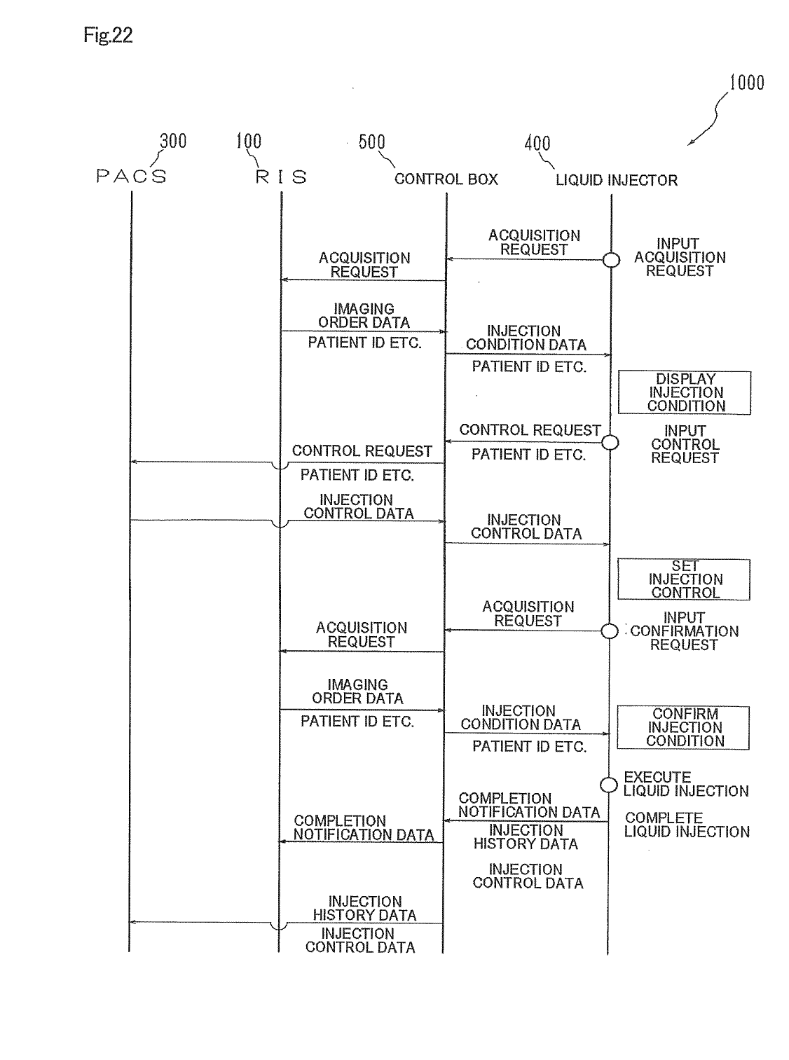

Once the operating icon of “acquisition request” is manually operated (step S3), the acquisition request is transmitted to the control box 500 as shown in FIG. 22. The control box 500 transfers the acquisition request received from the liquid injector 400, to the RIS 100.

The RIS 100 then returns the one of the imaging order data selected as above to the control box 500. The control box 500 returns, upon receipt of the imaging order data from the RIS 100, a part of the imaging order data to the liquid injector 400 as at least a part of the injection condition data.

More specifically, as already stated, the imaging order data includes the imaging job ID, the identification data of the CT scanner 200, the date and time of the start and finish of the image pickup, the patient ID and the name and body weight of the patient, the body part or region to be imaged, and the type and size of the syringe needle.

The control box 500 extracts the imaging job ID, the patient ID and the name and body weight of the patient, the body part or region to be imaged, and the type and size of the syringe needle, and so on out of the acquired imaging order data, and returns such data to the liquid injector 400 as the injection condition data.

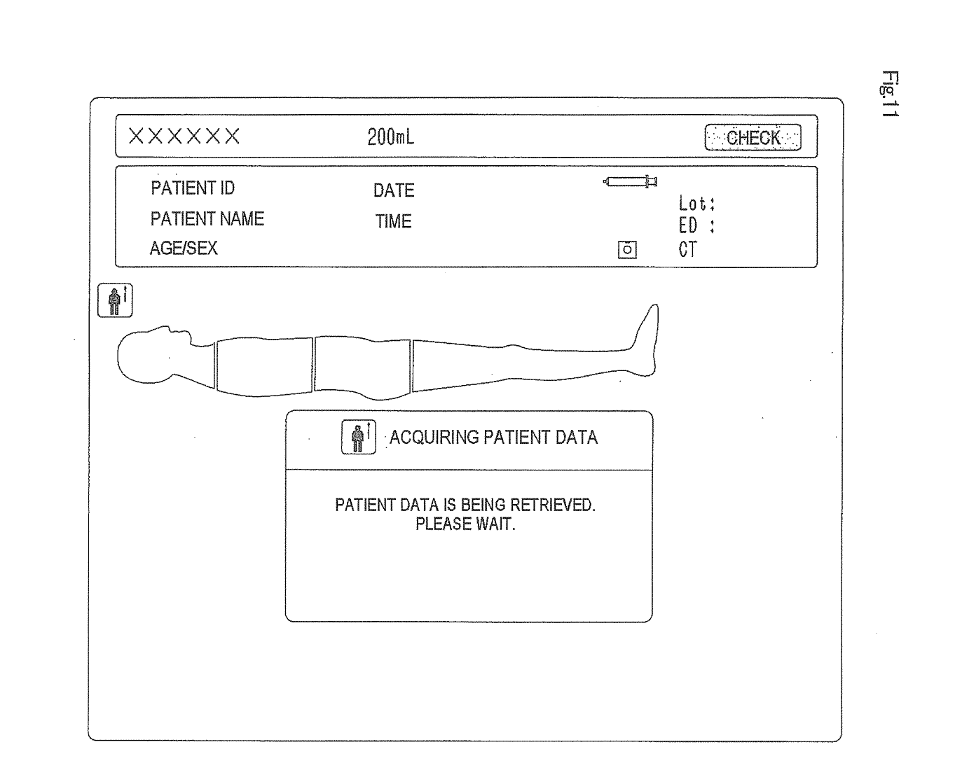

During such communication, the liquid injector 400 displays the guidance data indicating that the communication is being made, on the touch panel 403 and the head display 415, as shown in FIG. 11. Therefore, the operator can confirm at real time that the liquid injector 400 is executing the communication.

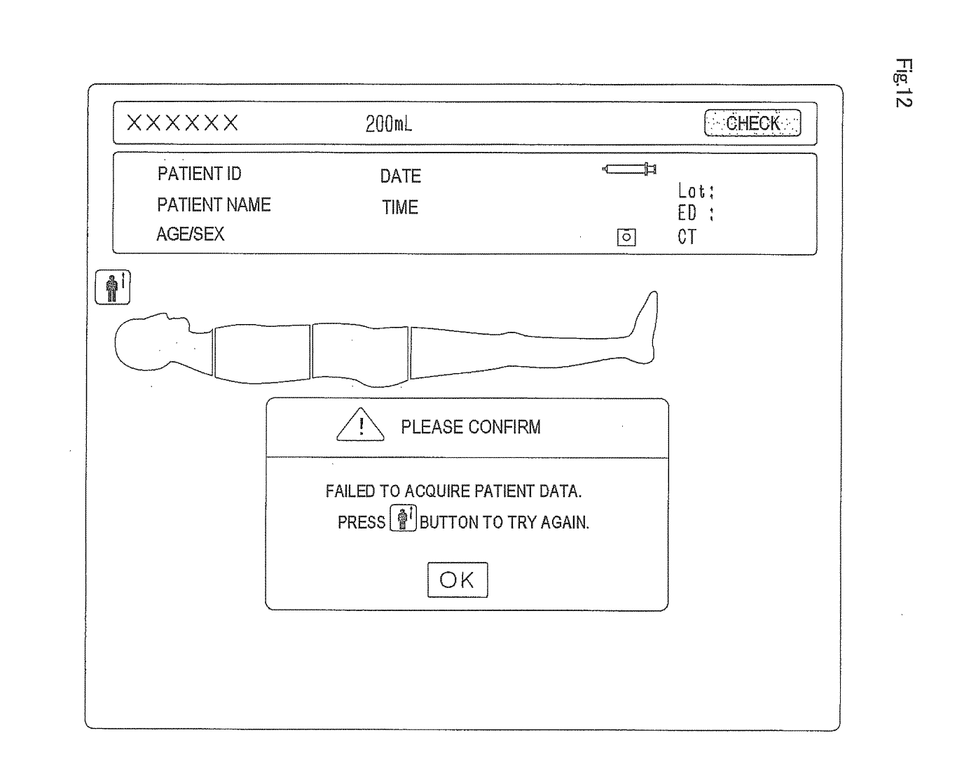

Also, in the case where the injection condition data cannot be acquired because of a communication error or the like, guidance data indicating the failure in acquiring the data is displayed on the touch panel 403 and the head display 415, as shown in FIG. 12. This enables the operator to immediately recognize the failure in data acquisition, and to take another step.

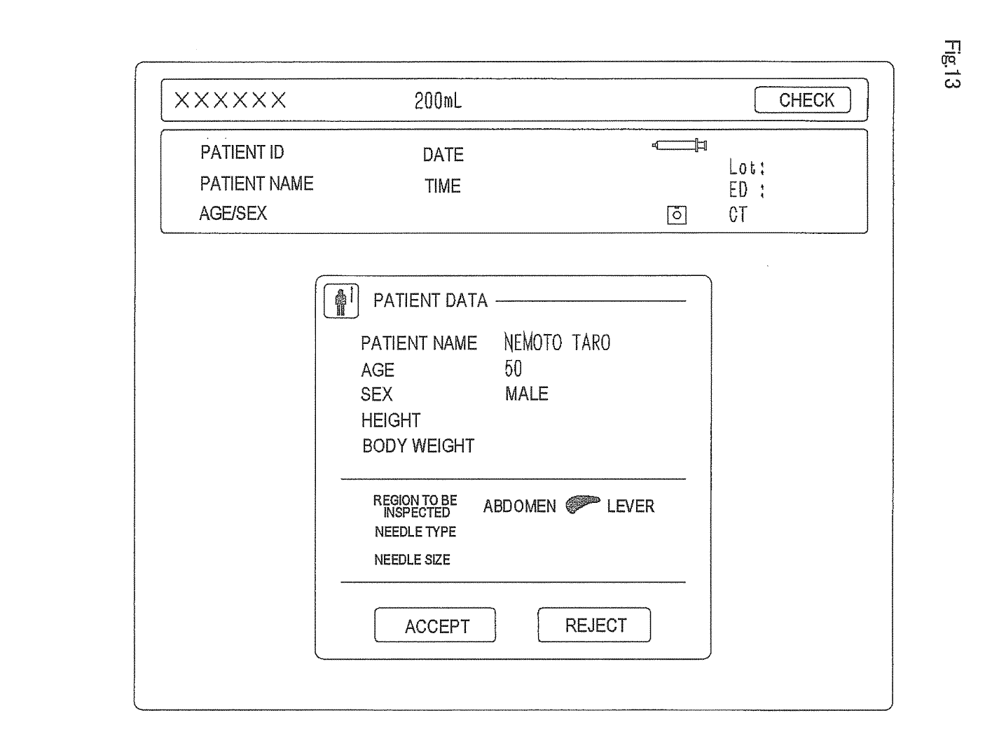

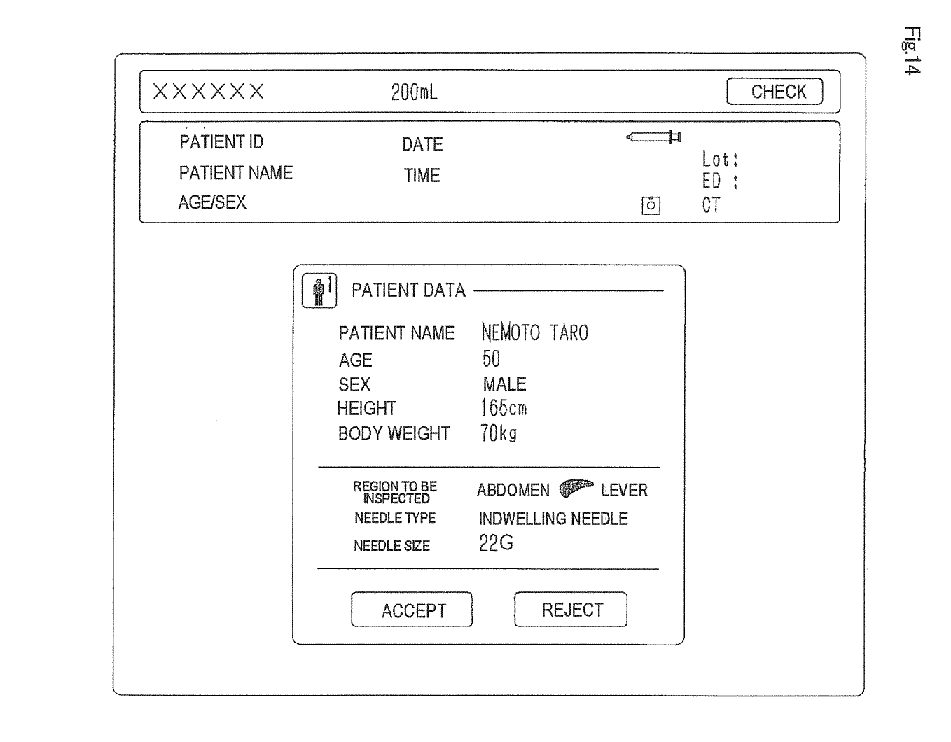

In the liquid injector 400, the injection condition data acquired from the control box 500 in response to the acquisition request (step S12) is displayed on the touch panel 403 and the head display 415, as shown in FIGS. 13 and 14 (step S13).

At the same time, the name and sex of the patient are also displayed as the injection condition data, based on which the operator can check the accordance between the injection condition data and the actual patient. Also, an operating icon for instructing whether to use the injection condition data as the condition for acquiring the injection control data is displayed on the touch panel 403 and the head display 415, together with the injection condition data displayed as above.

In the case of acquiring the injection control data based on the injection condition data confirmed by the operator, the operator touches the operating icon indicating “accept”. The liquid injector 400 sets, upon detecting such input, the patient ID and the body part or region to be imaged included in the injection control data as the condition for acquiring the injection control data.

In this process also, whether the RFID chip 810 is mounted in the liquid syringe 800 is confirmed (step S15) as stated above, and in the affirmative case the liquid condition data is acquired and displayed as shown in FIG. 15 (steps S16, S17).

Then the liquid injector 400 according to this embodiment transmits, upon receipt of the input of the acquisition of the injection control data through the injection control unit 401 (step S18), the patient ID and the body part or region to be imaged acquired from the imaging order data as the injection condition data as above to the control box 500 together with the acquisition request for the injection control data, as shown in FIG. 22 (step S19).

The control box 500 then transmits the patient ID and the body part or region to be imaged to the PACS 300 together with the acquisition request for the injection control data. The PACS 300 stores, as will be subsequently described in further details, accumulated data of the fluoroscopic image data, the injection history data, and the injection control data together with the patient ID and body part, with respect to each of the preceding imaging jobs.

Upon receipt of the acquisition request for the injection control data as above, the PACS 300 retrieves the injection control data that agrees with the patient ID and the body part or region to be imaged that have been received.

Upon retrieving the injection control data, the PACS 300 returns the injection control data to the control box 500, and the control box 500 in turn transfers the injection control data to the liquid injector 400.

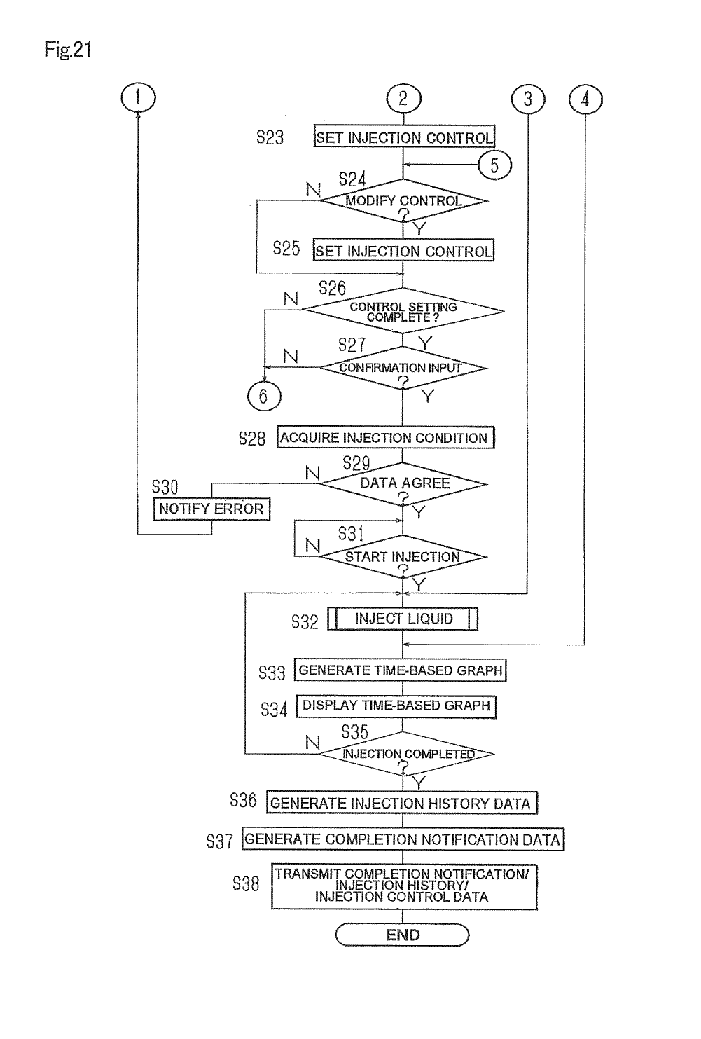

Upon receipt of the injection control data (step S21), the liquid injector 400 sets the received injection control data as the renewed injection control data as shown in FIG. 21 (step S23).

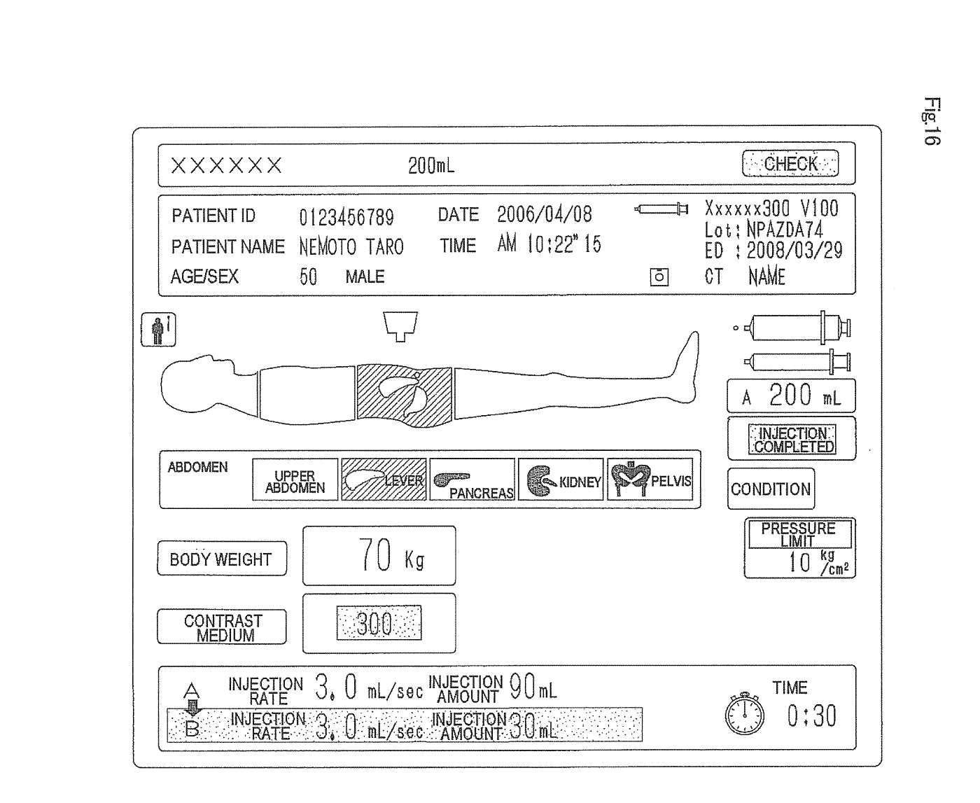

At this stage, the injection rate or total injection amount, constituting a part of the injection control data, are displayed on the touch panel 403 and the head display 415 together with the patient's name which is a part of the injection condition data, as shown in FIG. 16. Upon confirming the displayed injection control data, the operator can manually modify the injection control data if necessary (step S24, S25).

Here, even though the liquid injector 400 transmits the patient ID and the region to be imaged to the PACS 300 (step S19), in the case where the patient has not undergone the fluoroscopic image pickup with respect to the same body part or region to be imaged so far, naturally such injection control data that agrees with the patient ID or the region to be imaged is not retrieved by the PACS 300.

In this case the PACS 300 returns a message of “no data” to the control box 500, and the control box 500 transfer the message to the liquid injector 400.

Upon receipt the message of “no data” (step S20), the liquid injector 400 outputs a guidance text to such an effect that “Control data corresponding to this patient is not registered, manual setting is required” for display (step S22). In view of such guidance text, the operator should manually set the injection control data (step S24, S25).

The liquid injector 400 according to this embodiment is not yet ready to start the liquid injection, at the time of completing the setting of the injection control data by receiving the data from the PACS 300 or by manual operation (step S26).

Therefore, upon completion of the injection control data setting (step S26), the operator manually operates the final confirmation switch 414 of the injection head 410, right before starting the liquid injection.

Upon detecting the input of the final confirmation switch 414 (step S27), the liquid injector 400 causes the control box 500 to acquire the imaging order data again from the RIS 100, and acquires a part of the imaging order data as the injection condition data again from the control box 500, as in the first process (step S28). Then the patient ID specified in the injection control data and that in the injection condition data acquired again are compared (step S29).

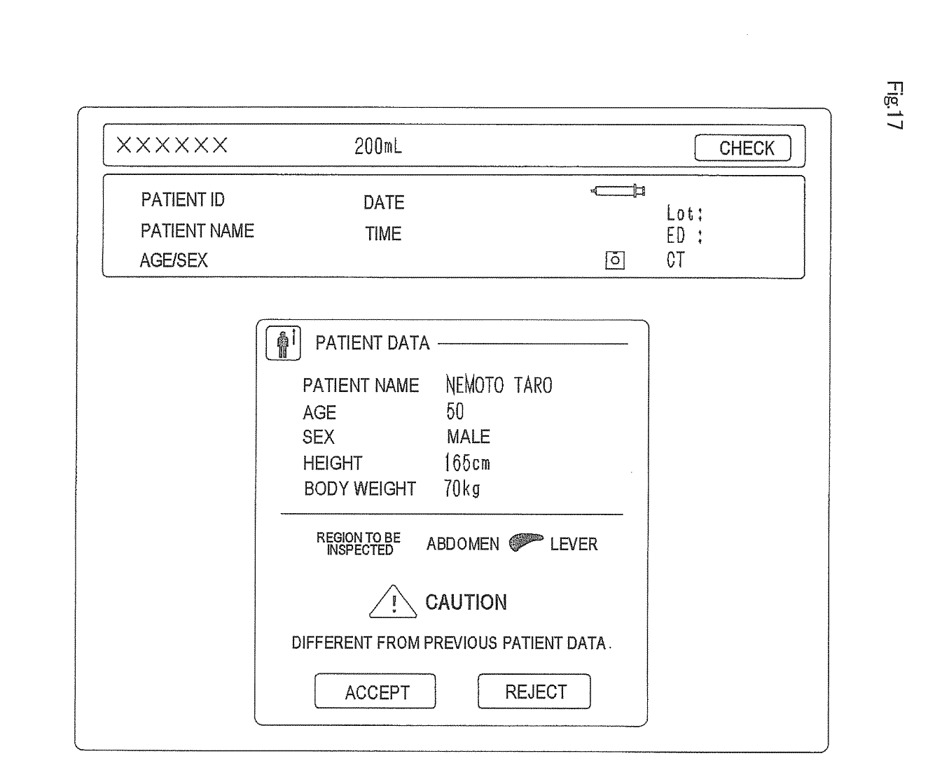

In the case where the patient ID does not agree, an error guidance urging confirmation such as “Different from previous patient data” is displayed on the touch panel 403 and the head display 415, for example as shown in FIG. 17 (step S30), together with the second injection condition data (not shown).

In this case, when the completion of confirmation is input through the touch panel 403 and the head display 415 for example, the screen returns to the initial state (step S3). Therefore, the injection cannot be started under the modified imaging order data.

On the other hand, once agreement between the patient ID specified in the injection control data and that in the injection condition data acquired again is confirmed (step S29), the liquid injector 400 becomes ready to accept the input of starting instruction through the touch panel 403 and the head display 415.

Once the starting instruction is input under such state (step S31), the syringe driving mechanism 412 is activated under control according to the injection control data, so that the contrast medium and physiological saline are properly injected to the patient (step S32).

In this process, the lapse of time is measured on a real time basis and the actual injection rate is detected, so that a feedback control is executed upon the syringe driving mechanism 412 such that the injection rate agrees with the injection control data.

Also, the time-based graph indicating the actual injection rate is generated on a real time basis (step S33), and is displayed on the touch panel 403 and the head display 415, for example with the injection control data (step S34).

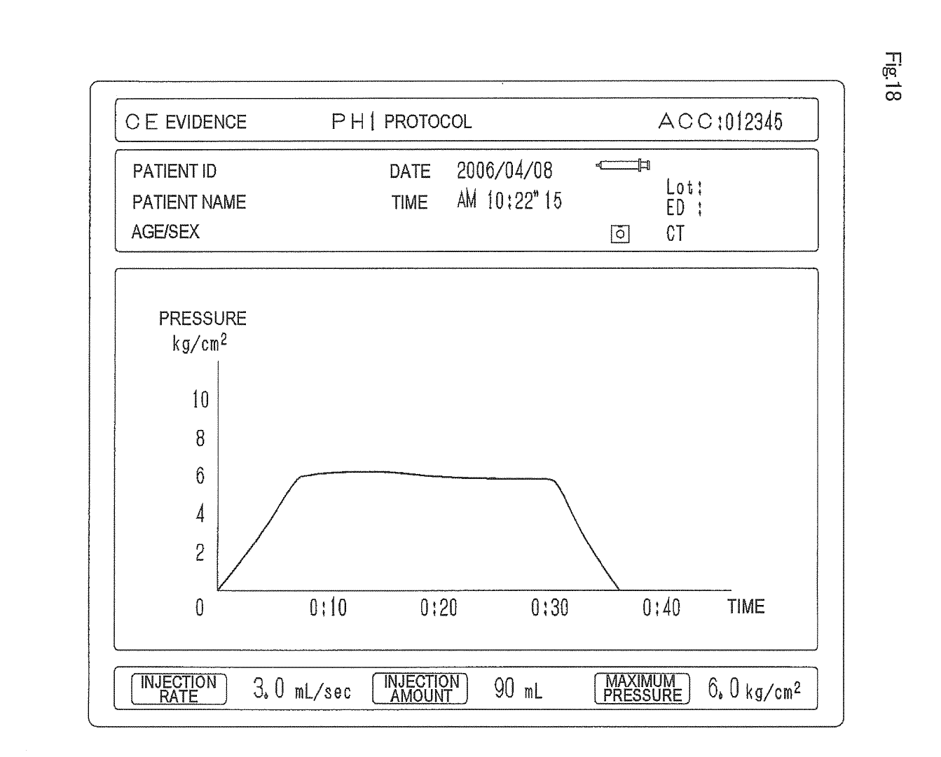

In the case where the injection control data is manually set without acquiring the injection condition data as above, the time-based graph is displayed together with the injection control data manually set, as shown in FIG. 18.

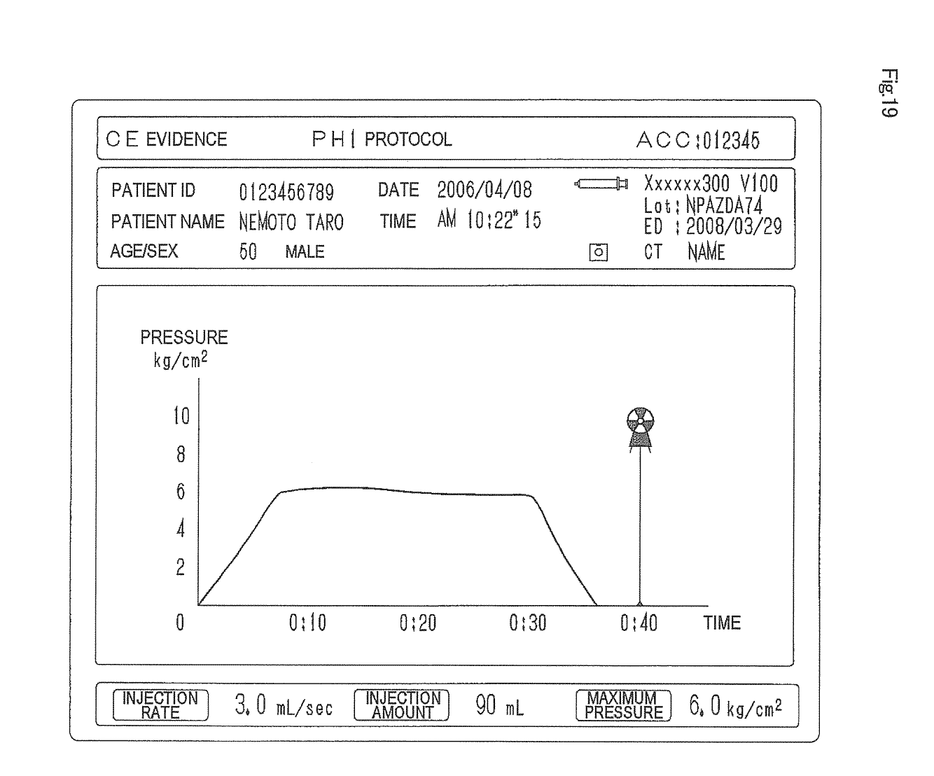

On the other hand, in the case where the injection control data is automatically set upon acquiring the injection condition data, the time-based graph is displayed with the injection control data automatically set and injection condition data, as shown in FIG. 19. In this case, further, a predetermined symbol indicating the starting time of the imaging acquired from the injection condition data is also displayed on the time-based graph.

Once the injection job is completed (step S35), the injection history data including the actual time-based graph is generated (step S36). The injection history data thus generated includes the image data of the time-based graph and the text data of the injection control data manually set, in the case where the injection control data is manually set without acquiring the injection condition data.

In the case where the injection condition data is acquired and the injection control data is automatically set, the injection history data includes the image data of the time-based graph and the text data of the injection control data and the injection condition data.

The text data includes, for example, the injection condition data including the injection job ID and patient ID, information on whether the injection condition data has been acquired from the RIS 100 or manually input, actual date and time of the start and finish of the injection, identification data of the liquid injector 400, the injection control data, and information on whether the injection control data has been acquired from the PACS 300 or manually input.

The liquid injector 400 also generates, upon completing the injection, completion notification data to which at least the injection job ID is allocated and which serves to notify of the completion (step S37). Upon completing the injection job, therefore, the liquid injector 400 transmits the completion notification data, the injection condition data and the injection control data to the control box 500 (step S38). At the same time, the liquid injector 400 allocates also at least the patient ID and the body part or region imaged, to the injection control data.

Then upon receipt of the completion notification data, the injection condition data and the injection control data from the liquid injector 400, the control box 500 transfers only the completion notification data to the RIS 100. The RIS 100 stores the completion notification data received, in association with the imaging order data via the injection job ID.

The control box 500 also transfers, upon receipt of the completion notification data, the injection condition data and the injection control data from the liquid injector 400, the completion notification data, the injection condition data and the injection control data to the PACS 300.

The PACS 300 stores the received injection history data and the injection control data, utilizing the imaging job ID as the index. To the injection control data thus stored, the patient ID and the body part or region imaged are also allocated.

In a normal operation, around the time when the liquid injector 400 completes the injection job as above, the imaging job by the CT scanner 200 is started. In this case, the operator inputs the start of the imaging job to the imaging control unit 210 of the CT scanner 200.

The imaging control unit 210 of the CT scanner 200 then transmits the acquisition request for the imaging order data to the RIS 100. The RIS 100 returns the imaging order data selected as above to the CT scanner 200.

Then the CT scanner 200 controls the action of the fluoroscopic imaging unit 201 according to the imaging order data received by the imaging control unit 210, so that the fluoroscopic image data is picked up.

Once the fluoroscopic imaging unit 201 thus picks up the fluoroscopic image data of the patient, the imaging control unit 210 allocates the fluoroscopic image data with the imaging order data. The imaging control unit 210 then transmits the fluoroscopic image data allocated with the imaging order data to the PACS 300.

The PACS 300 stores the fluoroscopic image data, utilizing the imaging job ID of the imaging order data as the index. When the operator is to review the fluoroscopic image data, the operator may for example manually operate the image viewer 600, to thereby read out the fluoroscopic image data from the PACS 300.

In this case, inputting for example the imaging job ID as the retrieval key causes the fluoroscopic image data corresponding to that imaging job ID to be read out from the PACS 300, and to be displayed on the display unit 602 of the image viewer 600.

At the same time, the injection history data is also read out from the PACS 300 based on the imaging job ID, and the injection history data can also be displayed on the display unit 602 of the image viewer 600, if need be.

From the injection history data thus displayed, it can be confirmed whether the whole injection control data for the injection job has been manually input, or partially acquired from the imaging order data, or partially acquired from the liquid condition data. In the case of acquisition from the imaging order data, the date and time of the first injection and the confirmation, and the acquired injection condition data and liquid condition data can also be confirmed.

In the fluoroscopic imaging system 1000 according to this embodiment, once the patient ID and the injection control data are input to the liquid injector 400 and the liquid injection is executed, the corresponding injection control data is registered in the PACS 300 with the patient ID.

When the same patient is to undergo the second and subsequent liquid injection job, the injection control data registered in the PACS 300 is acquired based on the patient ID to be input, and the injection control data thus acquired is set in the liquid injector 400.

Such arrangement eliminates the need to repeat the input of the same injection control data for the same patient, thereby alleviating the setting work. Moreover, since the injection control data for each patient is registered and acquired based on the patient ID as the index, erroneous setting of injection control data and injection of inappropriate liquid to the patient can be automatically prevented.

In particular, the injection control data is stored in the PACS 300 together with the patient ID and the body part or region to be imaged input to the liquid injector 400, so that the injection control data can be retrieved by the PACS 300 utilizing the patient ID and the body part or region to be imaged input to the liquid injector 400 as the index.

Accordingly, the injection control data cannot be acquired in the case where the body part or region to be imaged is different although the patient is the same, and hence inappropriate injection control data, for the same patient but for a different body part or region to be imaged, cannot be applied to the injection job.

Also, the liquid injector 400 according to this embodiment automatically selects the latest one out of a plurality of injection control data acquired, in the case where the plurality of injection control data acquired has been acquired from the PACS 300 based on the patient ID.

Therefore, the liquid injector 400 automatically selects one injection control data even in the case where the same patient has undergone the fluoroscopic image pickup a plurality of times with respect to the same body part of region to be imaged. Also, since the latest injection control data is selected, the optimal injection control data can be automatically set even though the disease of the patient is progressing.

Further, the liquid injector 400 according to this embodiment also accepts new setting and modification of the setting through manual operation. Accordingly, in the case where the injection control data acquired from the PACS 300 is not appropriate, such injection control data can be manually modified, or the entirety of the injection control data can be input by manual operation.

Especially, in the case where the body weight of the patient has been fluctuating because of the progress of the disease, it is preferable to modify the injection control data according to the actual body weight. Since previous injection control data can be utilized even in such a case, the simplicity in inputting the injection control data can still be maintained.

Further, in the fluoroscopic imaging system 1000 according to this embodiment, the RIS 100 stores the imaging order data in which the patient ID is specified, and the CT scanner 200 picks up the fluoroscopic image data of the patient according to the imaging order data.

The liquid injector 400 acquires the patient ID as a part of the imaging order data from the RIS 100. Accordingly, the patient ID and the body part or region to be imaged necessary for acquiring the injection control data can also be automatically input to the liquid injector 400. Such arrangement alleviates the operational burden on the operator for inputting the patient ID, and prevents erroneous input thereof.

Still, since the imaging order data has been indispensible for the CT scanner 200 and hence available from the past, at least a part of the injection condition data can be automatically set, without the need to create new data.

Also, the liquid injector 400 according to this embodiment acquires the patient ID again from the RIS 100 once the final confirmation is input right before the injection, and checks the agreement between the previously acquired patient ID and the newly acquired patient ID.

The liquid injector 400 does not execute the liquid injection based on the injection control data unless such agreement is confirmed, and therefore the injection according to inappropriate injection control data can be easily and surely prevented.

For example, in the case where the imaging order data has been modified or deleted because of a sudden change of the image pickup schedule, the injection based on the first imaging order data is inhibited from being executed.

Also, since the liquid injector 400 notifies the operator to the effect that the imaging order data is not in accordance, the operator does not fail to recognize and confirm the modification of the imaging order data.

In a normal injection job utilizing the liquid injector 400, the injection control data is set through the injection control unit 401 located away from the injection head 410 as above, and the operator finally confirms, upon completing the setting, the condition of the liquid syringe 800 and the patient at the position close to the injection head 410.

Whereas, in the liquid injector 400 according to this embodiment, the final confirmation switch 414 for acquiring the imaging order data again for confirmation the agreement right before starting the injection is provided on the injection head 410. Therefore, the inputting operation for acquiring the imaging order data again for confirming the agreement can be smoothly executed at the time of final confirmation at the position close to the injection head 410, which has been mandatory from the past.

Also, the liquid injector 400 according to this embodiment does not permit the start of the injection unless the patient ID of the imaging order data is acquired again and confirmed, even though the injection control data corresponding to the patient ID of the imaging order data is once acquired.

However, in the case where the entirety of the injection control data is manually input, the injection can be started once setting of the injection control data has been completed. Therefore, inhibition and permission of the injection can be properly controlled cased on a simple condition.

Also, during the injection according to the injection control data, the time-based graph is displayed on a real time basis, and hence the operator can confirm the injection status on a real time basis.

In the case where the injection control data is acquired from the PACS 300 and set, in particular, the details of the data are displayed with the time-based graph, and therefore the operator can constantly confirm the details of the injection control data.

Further, in the liquid injector 400 according to this embodiment, in the case where the imaging order data is acquired from the RIS 100, the starting time of the imaging job is displayed on the time-based graph according to the imaging order data.

Accordingly, the operator can confirm the relationship between the progress of the injection and the injection starting time on a real time basis. Besides, since an exclusive logo mark is used to indicate the injection starting time, the operator can intuitively confirm the status.

Further, in the fluoroscopic imaging system 1000 according to this embodiment, the injection history data and the injection control data are also stored in association with the fluoroscopic image data stored in the PACS 300, as already stated.

Accordingly, at the time of viewing the fluoroscopic image data for example, the injection history data and the injection control data can also be confirmed. Such arrangement enables the operator viewing the fluoroscopic image data to even confirm how the liquid was injected to the patient during the fluoroscopic image data pickup.

Also, since the injection control data is stored in the PACS 300 together with the patient ID and so on, the liquid injector 400 can acquire the injection control data utilizing the patient ID etc. as index and apply the data to the injection job.

Besides, whereas the fluoroscopic image data and the injection history data are associated via the imaging job ID as stated above, the imaging job ID is acquired by the liquid injector 400 as the imaging order data, when automatically setting the injection control data.

In other words, the imaging order data acquired by the liquid injector 400 from the RIS 100 through the control box 500 can be utilized for both the acquisition of the injection control data and generation of the injection history data.

Also, in the case where the injection job of the contrast medium turns to be suspicious, the injection history data and the injection control data can be employed as the evidence, because the injection history data and the injection control data can also be confirmed together with the fluoroscopic image data.

In particular, the units 100 to 600 of the fluoroscopic imaging system 1000 according to this embodiment mutually execute the data communication in accordance with the DICOM standards. Since it is difficult to falsify the communication data according to DICOM, the injection history data and the injection control data have high admissibility as evidence.

Moreover, the completion notification data of the injection job is transmitted from the liquid injector 400 through the control box 500 to the RIS 100, to be stored therein. Since the RIS 100 stores the completion notification data in association with the imaging order data, the RIS 100 can notify the CT scanner 200, for example, of the time of the start and finish of the liquid injection, together with the imaging order data.

In this case, the operator engaged with the CT scanner 200 can refer to the time of the start and finish of the liquid injection, and hence the operator can adjust the starting time of the image pickup according to the injection time.

Besides, since the injection condition data and the liquid condition data automatically acquired are displayed as above, the operator can easily and surely confirm whether the injection condition data and the liquid condition data are appropriate.

Moreover, the name and sex of the patient as part of the injection condition data, and product name as part of the liquid condition data are also displayed. Such arrangement enables the operator to easily and surely confirm the agreement between the injection condition data and the actual patient, and whether the liquid to be used is appropriate.

The acquisition of the injection condition data including the patient ID and so forth is executed through manipulation of the exclusive icon composed of such a logo as “i” and a human body icon. Such arrangement enables the operator to intuitively execute the acquisition of the injection condition data.

Also, the liquid condition data acquired from the RFID chip 810 is displayed with a predetermined “RFID” logo mark. Such arrangement enables the operator to intuitively confirm that the liquid condition data displayed has been acquired from the RFID chip 810.

Besides, the foregoing display also appears on the head display 415 of the injection head 410, in addition to the touch panel 403 on the injection control unit 401 of the liquid injector 400. Therefore, the operator can confirm the injection condition data and the liquid condition data even while working close to the injection head 410.

It is to be noted that the present invention is in no way limited to the foregoing embodiment, but allows various modifications within the scope of the present invention. To cite some examples, although the embodiment exemplifies the case where the injection control data utilized by the liquid injector 400 for the injection job is registered in the PACS 300 together with the injection history data and the patient ID, and the injection control data is acquired by the liquid injector 400 from the PACS 300 utilizing the patient ID as index, to be utilized for the injection job.

However, the injection history data generated based on the injection job in the liquid injector 400 may be registered in the PACS 300 with the patient ID etc., and such injection history data may be acquired by the liquid injector 400 from the PACS 300 utilizing the patient ID etc. as index, to be utilized for the injection job as the injection control data.

In this case, although the injection control data has to be generated from the injection history data in the liquid injector 400, registering the injection history data in the PACS 300 allows skipping the registration of the injection control data.

According to the foregoing embodiment, the injection control data and the injection history data are registered in the PACS 300, which is independent from the liquid injector 400. However, the injection control data and the injection history data may be registered in the liquid injector 400 (not shown).

According to the foregoing embodiment, the body part or the region to be imaged is also input to the liquid injector 400 as a part of the predetermined injection condition data associated with the liquid injection condition, and the PACS 300 stores the injection control data together with not only the patient ID but also the body part or region to be imaged, so that the liquid injector 400 can acquire the injection control data corresponding not only to the patient ID but also to the body part or region to be imaged, from the PACS 300.

However, the product ID, ingredients, chemical classification, or concentration of components of the liquid can also be utilized as the injection condition data. In this case, the injection condition data may also be acquired by the liquid injector 400 from the imaging order data registered in the RIS 100, or manually input to the liquid injector 400, or may be acquired by the liquid injector 400 from the RFID chip 810 in the liquid syringe 800.

Here, in a normal operation the fluoroscopic image data of the CT scanner 200 and the MRI equipment (not shown) are separately stored, however in the case where the fluoroscopic image data is mixedly stored in a single PACS 300, the format distinction of the image data can also be utilized as the injection condition data.

According to the foregoing embodiment, the injection control data acquired from the PACS 300 by the liquid injector 400 can be manually modified if necessary. However, the injection control data acquired from the PACS 300 by the liquid injector 400 as above may be automatically adjusted according to the injection condition data.

As such injection condition data, the body weight of the patient, the ingredients, and composition concentration etc. of the liquid can be utilized. For example, in the case where the patient's body weight allocated to the past injection control data acquired from the PACS 300 by the liquid injector 400 is 50 kgs., while the current body weight input in the liquid injector 400 is 60 kgs., it is preferable to increase the injection rate and total injection amount in the injection control data by 20% (=60/50).

Likewise, in the case where the component concentration of the liquid allocated to the past injection control data is 10%, while the current component concentration input in the liquid injector 400 is 20%, it is preferable to decrease the injection rate and total injection amount in the injection control data to 50% (=10/20).

Also, the injection condition data to be utilized for the automatic adjustment of the injection control data can also be acquired by the liquid injector 400 from the imaging order data registered in the RIS 100, or manually input to the liquid injector 400, or may be acquired by the liquid injector 400 from the RFID chip 810 in the liquid syringe 800.

According to the foregoing embodiment, displaying the product ID, ingredients and chemical classification of the liquid acquired by the liquid injector 400 from the RFID chip 810 mounted in the liquid syringe 800 enables the operator to confirm the data on the liquid.

However, the PACS 300 may also store the product ID of the liquid syringe 800 together with the injection control data, and the liquid injector 400 may acquire the product ID the ingredients and chemical classification of the liquid together with the injection control data, so that the product ID acquired with the injection control data and that acquired from the liquid syringe 800 are compared, and that a warning is output for notification in the case of disagreement (not shown).

Likewise, the PACS 300 may also store the ingredients and chemical classification of the liquid together with the injection control data, and the liquid injector 400 may acquire the ingredients and chemical classification from the PACS 300 together with the injection control data, so that the ingredients and chemical classification acquired with the injection control data and those acquired from the liquid syringe 800 are compared, and that a warning is output for notification in the case of disagreement.

With the fluoroscopic imaging system thus arranged, such medical malpractice as injecting a liquid different from the one previously used can be prevented. In particular, since the product ID and so on are acquired from both the previous injection control data and the liquid syringe 800 to be newly employed, the medical malpractice as injecting a liquid different from the previous one is automatically and assuredly prevented.

Also, the imaging order data stored in the RIS 100 may include, in addition to the patient ID, an NG ID representing the product ID of a liquid inappropriate for injection, with respect to each patient, and the liquid injector 400 may acquire the product ID from the RFID chip 810 in the liquid syringe 800 and the NG ID from the imaging order data, for comparison between the product ID acquired from the liquid syringe 800 and the NG IG acquired from the imaging order data, so that in the case of agreement such a warning message as “unusable for this patient” is displayed on the touch panel, and that the liquid injection mechanism is disabled.

With such arrangement, for example in the case where the liquid to be injected to the patient is unsuitable because of a personal reason of the patient such as risk of side effect, despite that the type of the liquid such as contrast medium for CT scanning is correct, the liquid injector 400 can automatically detect such fact and output a warning to the operator. Accordingly, the operator can promptly recognize the situation and take a necessary step such as replacing the liquid.

In particular, once the liquid syringe 800 is loaded on the injection head 410 the product ID is automatically acquired, and once the imaging order data is input as the injection control data the NG ID is also automatically acquired. Such arrangement eliminates the need to perform an exclusive procedure for acquisition or comparison, thereby enabling easy and assured comparison of the product ID and the NG ID.

Likewise, the imaging order data stored in the RIS 100 may include, in addition to the patient ID, an NG ingredient and NG chemical classification representing the ingredient and chemical classification inappropriate for injection with respect to each patient, and the liquid injector 400 may acquire the ingredient and chemical classification from the RFID chip 810 in the liquid syringe 800 and also the NG ingredient and NG chemical classification from the imaging order data, for comparison therebetween, so that in the case of agreement between the ingredient and the NG ingredient, or between the chemical classification and the NG chemical classification, a warning message is displayed on the touch panel, and that the liquid injection mechanism is disabled.

Such arrangement allows managing the possibility of injection to the patient in terms of the ingredients and chemical classification. Accordingly, for example in the case of using a brand new liquid, the possibility can be correctly ascertained.

Further, the RFID chip 810 may contain, as a part of the liquid condition data, the applicable job type according to the imaging job, and the type of the job may be specified in the imaging order data, so that the liquid injector 400 compares the applicable job type acquired from the RFID chip 810 and the job type specified in the injection condition data, and outputs a warning in the case of disagreement.

With such arrangement, such medical malpractice as injecting a contrast medium for MRI to the patient from whom the fluoroscopic image is to be shot by a CT scanner 200 can be prevented. On the contrary, the applicable liquid type according to the imaging job may be specified in the imaging order data, so that the liquid injector 400 compares the liquid type acquired from the RFID chip 810 and the applicable liquid type specified in the injection condition data, and outputs a warning in the case of disagreement.

According to the foregoing embodiment, the acquisition of the patient ID and the region to be imaged by the liquid injector 400 from the imaging order data registered in the RIS 100 exempts the operator from inputting the patient ID and so on to the liquid injector 400, thereby preventing an erroneous input.

However, a part or all of the patient ID, the region to be imaged and so forth may be input to the liquid injector 400. Also, the fluoroscopic imaging system 1000 may include a patient management medium (not shown) with respect to each patient, in which an RFID chip containing at least the patient ID is mounted, so that the liquid injector 400 may acquire the patient ID from the RFID chip in the patient management medium.

Such patient management medium may be realized in a form of, for example, an electronic medical record with the RFID chip mounted thereon, or a managing arm band to be attached to the patient's arm (not shown).

Such arrangement exempts the operator from inputting the patient ID, thereby preventing an erroneous input. Also, the injection condition data such as the region to be imaged and the body weight may be registered in the RFID chip of the patient management medium, so that the liquid injector 400 acquires such data and utilizes for the automatic adjustment of the injection control data.

According to the foregoing embodiment, the acquisition of the patient ID in the imaging order data in the RIS 100 by the liquid injector 400 again right before the injection enables coping with the modification of the imaging order data.

However, liquid injector 400 may also confirm the agreement between the patient ID acquired from the imaging order data in the RIS 100 and the patient ID acquired from the RFID chip in the patient management medium, so as to inhibit the action control of the syringe driving mechanism 412 until the agreement is confirmed, and to output a predetermined warning for reconfirmation in the case of disagreement.

According to the foregoing embodiment, in the case where a plurality of injection control data corresponding to the patient ID provided by the liquid injector 400 is registered in the PACS 300, the plurality of injection control data is transmitted from the PACS 300 to the liquid injector 400, so that the liquid injector 400 selects the latest one of the injection control data.

However, the PACS 300 may select the latest one of the injection control data and transmit such data to the liquid injector 400. In this case, although the PACS 300 is required to have an additional exclusive process, the amount of data to be transmitted can be reduced, so as to prevent congestion.

Also, the plurality of injection control data transmitted from the PACS 300 may be displayed on the liquid injector 400 in a form of a listing, so that one of the listed injection control data is selected. In this case, the operator can select the desired optimal injection control data. Further, the liquid injector 400 may select one out of the plurality of injection control data transmitted from the PACS 300 according to a predetermined condition, for example that the body weight is closest.

According to the foregoing embodiment, the entirety of the imaging order data managed by the RIS 100 is acquired by the control box 500, and the liquid injector 400 acquires a part of the imaging order data from the control box 500 as the injection condition data. However, the liquid injector 400 may acquire the entirety of the imaging order data as the injection condition data.

According to the foregoing embodiment, the RIS 100 is of the push-type, and the control box 500 acquires the proper imaging order data at a predetermined timing. However, the RIS 100 may be of the pull-type.

In the latter case, the CT scanner 200 transmits the acquisition request for the imaging order data to the RIS 100 with at least an order retrieval key. Then the RIS 100 selects one of the plurality of imaging order data according to the acquisition request and the order retrieval key received from the CT scanner 200, and returns the selected data.

The control box 500 then transmits to the RIS 100 the acquisition request for the imaging order data received from the liquid injector 400. The RIS 100 returns one of the imaging order data selected according to the acquisition request received from the control box 500.

Alternatively, the RIS 100 may return a plurality of imaging order data according to the acquisition request received from the CT scanner 200. In this case, the CT scanner 200 accepts an operation of selecting one of the plurality of imaging order data returned, and notifies the RIS 100 of the selected imaging order data.

The RIS 100 may also retrieve a part of the plurality of imaging order data based on the acquisition request and the order retrieval key received from the CT scanner 200, and return the retrieved data. The CT scanner 200 accepts an operation of selecting one of the imaging order data returned, and notifies the RIS 100 of the selected imaging order data.

Once the control box 500 transmits the acquisition request for the imaging order data to the RIS 100, the RIS 100 returns the one of the imaging order data notified of by the CT scanner 200, according to the acquisition request received from the control box 500.

Such arrangement allows the control box 500 to acquire the proper imaging order data despite that the RIS 100 is of the pull-type, and to allocate the imaging job ID and so on to the injection history data.

According to the foregoing embodiment, the control box 500 acquires the imaging order data from the RIS 100. However, the RIS 100 and the CT scanner 200 may be connected via the control box 500, so that the control box 500 may acquire the imaging order data which is transmitted from the RIS 100 to the CT scanner 200.

Also, the control box 500 may be connected to the CT scanner 200 without being connected to the RIS 100, and may acquire the imaging order data from the CT scanner 200.

In this case, for example, the control box 500 may transfer the acquisition request received from the liquid injector 400 to the CT scanner 200, and the CT scanner 200 may return the imaging order data according to the acquisition request received from the control box 500.

Alternatively, the CT scanner 200 may accept an operation of selecting one of the plurality of imaging order data returned from the pull-type RIS 100, to thereby transfer the selected imaging order data to the control box 500.

Also, the control box 500 may be connected to the RIS 100 and the CT scanner 200, so that the first imaging order data may be acquired from the RIS 100, and the imaging order data for confirmation may be acquired from the CT scanner 200.

According to the foregoing embodiment, the injection history data and the injection control data generated in the liquid injector 400 are stored in the PACS 300 together with the fluoroscopic image data generated in the CT scanner 200.

However, the injection history data and the injection control data may be transmitted from the liquid injector 400 to the RIS 100 through the control box 500, so that the RIS 100 may store the injection history data and the injection control data. In this case, the RIS 100 can manage the imaging order data, the injection history data, and the injection control data in mutual association via the job ID or the like.

Even in this case, the fluoroscopic image data registered in the PACS 300 is also allocated with the job ID of the imaging order data, and hence the fluoroscopic image data can be associated with the injection history data and the injection control data.

According to the foregoing embodiment, the entirety of the imaging order data is allocated to the fluoroscopic image data, when stored in the PACS 300. However, only the imaging job ID of the imaging order data may be allocated to the fluoroscopic image data.

Even in this case, the fluoroscopic image data can be associated with the injection history data and the injection control data via the imaging job ID, and therefore the imaging order data can be read out from the RIS 100 with the imaging job ID.

Alternatively, only the imaging job ID of the imaging order data may be allocated to the fluoroscopic image data, and the entirety of the imaging order data may be allocated to the injection history data and the injection control data, and also the imaging order data may be divided into portions to be allocated to the fluoroscopic image data, and to each of the injection history data and the injection control data. Also, the entirety of the display image on the touch panel 403 and the head display 415 of the liquid injector 400 may be included in the injection history data.

The foregoing embodiment only exemplifies the case where the injection condition data is set in the liquid injector 400. However, the injection condition data may be notified from the liquid injector 400 to the control box 500, and then from the control box 500 to the RIS 100. In the latter case, the injection condition data may be notified from the RIS 100 to the CT scanner 200, together with the imaging order data.

Such arrangement allows the person operating the CT scanner 200 to refer to the injection condition data, and therefore to adjust the imaging action according to the injection condition data. Further, automatic adjustment of the imaging action can also be executed according to the injection condition data acquired by the imaging control unit 210 of the CT scanner 200.

According to the foregoing embodiment, the liquid injector 400 completes the injection history data before transmitting to the control box 500. However, the liquid injector 400 may transmit the injection history data in divided portions to the control box 500, so that the control box 500 integrates the injection history data.

More specifically, the liquid injector 400 may transmit the injection condition data and the starting date and time to the control box 500 upon starting the injection, the injection rate and so on time after time during the injection, and the finishing date and time upon completing the injection. In this case, the control box 500 can complete the injection history data from various data accumulated during the period from the start of the injection to the finish thereof.

According to the foregoing embodiment, the respective units 100 to 600 mutually perform the data communication according to DICOM standard which is difficult to falsify, thereby securing high admissibility of the injection history data as evidence. However, the liquid injector 400 may generate the injection history data in another data format that is difficult to falsify, such as the Portable Document Format (PDF).

Likewise, the control box 500 may convert the injection history data received from the liquid injector 400 in the Joint Photographic Coding Experts Group (JPEG) format into the PDF format. Further, the liquid injector 400 and the control box 500 may be connected to what is known as the Internet, so as to acquire an electronic signature and allocate the injection history data with the same.

According to the foregoing embodiment, the head display 415 is directly attached to the injection head 410 so as to extend downward from a rear portion of the left side thereof, which is closer to the operator. However, the head display 415 may be attached to any position as long as the operation of the injection head 410 is not disturbed and the screen display can be confirmed.

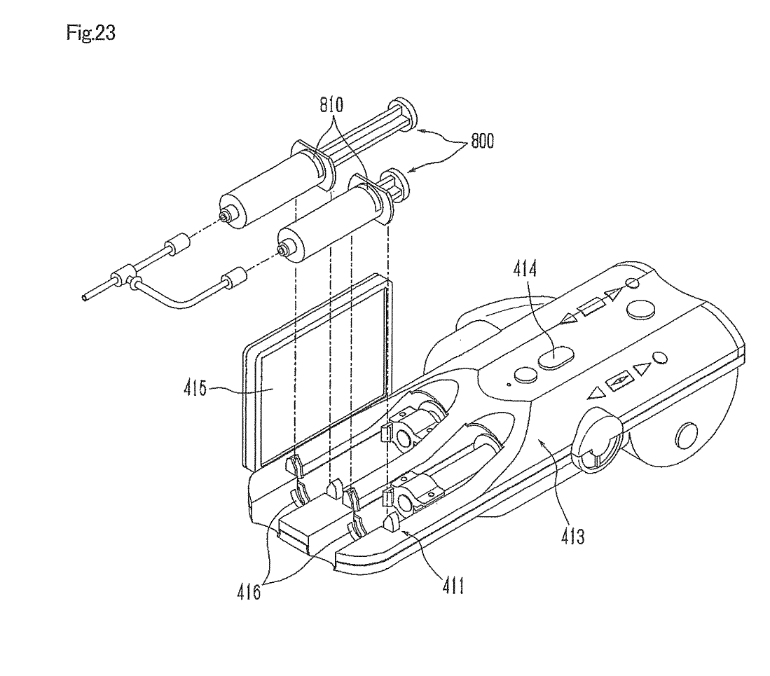

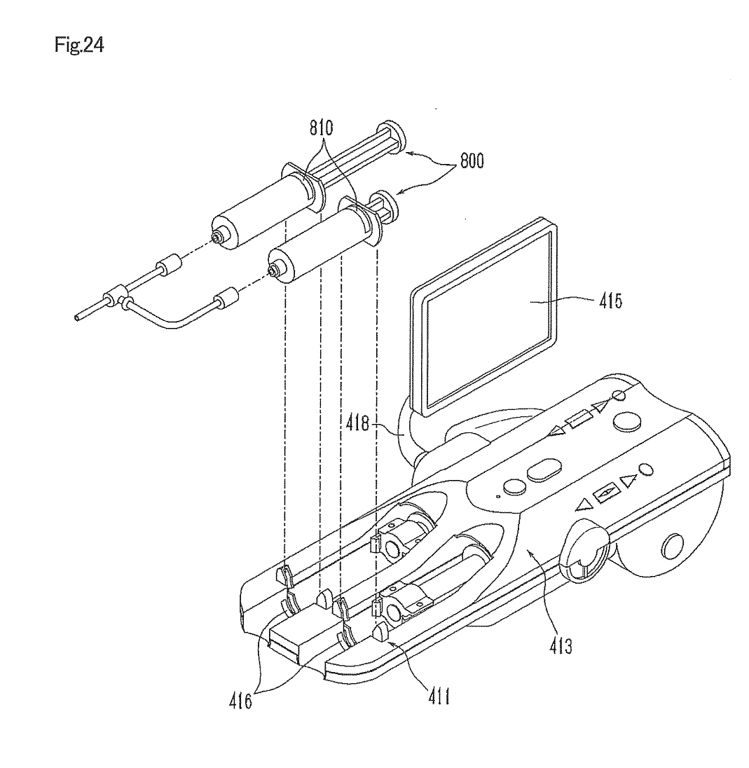

For example, the head display 415 may be attached to the right side or a forward portion of the injection head 410, or so as to extend upward therefrom, as shown in FIG. 23. Also, as shown in FIG. 24, the head display 415 may be pivotably mounted on the injection head 410 via a movable arm 418 or the like.

According to the foregoing embodiment, the liquid injector 400 utilizes a pair of liquid syringes 800 to inject the contrast medium and physiological saline to the patient. However, the liquid injector may utilize a single liquid syringe to inject the contrast medium and physiological saline to the patient (not shown).

Further, according to the foregoing embodiment, the CT scanner 200 serves as the imaging diagnostic apparatus, and the liquid injector 400 injects the contrast medium for CT scanning as the medical liquid. However, the imaging diagnostic apparatus may be constituted of a MRI equipment, a PET equipment, or an ultrasonic diagnostic equipment, and the liquid injector may inject the contrast medium prepared exclusively for such equipments.

Further, according to the foregoing embodiment, the CT scanner 200 and the liquid injector 400 are independently activated on a stand-alone basis. However, the CT scanner 200 and the liquid injector 400 may work in correlation to perform various actions, through data communication.

Still further, according to the foregoing embodiment, the fluoroscopic imaging system 1000 includes one each of the respective units, for the sake of explicitness of the description. However, in a large-scale hospital or the like, each of a plurality of fluoroscopic imaging systems may include one each of the RIS 100, the CT scanner 200, the liquid injector 400, and the control box 500, and the plurality of fluoroscopic imaging systems may share the PACS 300 and the image viewer 600 (not shown). In such case also, the hardware such as the RIS 100, the PACS 300, and the image viewer 600 may be prepared in a plurality of numbers and connected in parallel (not shown).

Still further, according to the foregoing embodiment, the fluoroscopic image data, the injection history data, and the injection control data are stored in a single unit of the PACS 300. However, the hardware that stores the fluoroscopic image data, the injection history data, and the injection control data may be independently prepared and connected via the communication network.

Still further, according to the foregoing embodiment, the RIS 100, the CT scanner 200, the PACS 300, the liquid injector 400, the control box 500, and the image viewer 600 are separately constructed and mutually connected via the communication network 701 to 706.

However, the respective units 100 to 600 may be integrally constructed in various combinations. To cite a few examples, the injection control unit 401 of the liquid injector 400 and the control box 500 may be integrally constituted; the RIS 100 and the PACS 300 may be added to such combination to thereby form a unified structure; and the PACS 300 and the image viewer 600 may be integrally constituted.

Also, the control box 500 may be unified with the RIS 100 and the PACS 300, and the control box 500, the PACS 30, and the image viewer 600 may be integrally constituted.

Further, the imaging control unit 210 of the CT scanner 200, the RIS 100, and the control box 500 may be integrally constituted; the imaging control unit 210 of the CT scanner 200, the PACS 300, and the control box 500 may be integrally constituted; and the image viewer 600 may be added to thereby form a unified structure.

Further, the image viewer 600 and the PACS 300 may be integrally constituted, and the control box 500 and the imaging control unit 210 of the CT scanner 200 may be added to thereby form a unified structure.

Still further, according to the foregoing embodiment, the computer unit works according to the computer program, to thereby logically realize the respective units 100 to 600 to perform the assigned functions.

However, it is also possible to set up the respective units as individually independent hardware, or some units as hardware and the others as software.

Naturally, the foregoing embodiment and the plurality of variations may be combined, unless contradiction arises. Further, although the foregoing embodiment and variations represent the specific structure of the respective constituents, such structure may be modified in various manners provided that the intended function according to the present invention is satisfied.

1.-33. (canceled)

34. A liquid injector for use in a fluoroscopic imaging system including an imaging diagnostic apparatus that picks up fluoroscopic image data of a patient, and said liquid injector that injects a medical liquid to said patient from whom said fluoroscopic image data is to be picked up, comprising:
a data input unit that acquires a patient ID of an individual patient;
a liquid injection mechanism that executes injection of said medical liquid;
an injection control unit that controls an action of a liquid injection mechanism based on injection control data;
a data registration unit that registers said injection control data utilized for controlling said action, together with said patient ID;
a data acquisition unit that acquires said injection control data corresponding to said acquired patient ID, from said data registration unit; and
a control setting unit that sets said acquired injection control data in said injection control unit.

35. A liquid injector for use in a fluoroscopic imaging system including an imaging diagnostic apparatus that picks up fluoroscopic image data of a patient, and said liquid injector that injects a medical liquid to said patient from whom said fluoroscopic image data is to be picked up, comprising:
a data input unit that acquires a patient ID of an individual patient;
a liquid injection mechanism that executes injection of said medical liquid;
an injection control unit that controls an action of a liquid injection mechanism based on injection control data;
a history generation unit that generates injection history data including an action history of said liquid injection mechanism corresponding to said injection control data;
a data registration unit that registers said generated injection history data together with said patient ID;
a data acquisition unit that acquires said injection history data corresponding to said acquired patient ID, from said data registration unit; and
a control setting unit that sets said acquired injection history data as said injection control data, in said injection control unit.

36. A liquid injector for use in a fluoroscopic imaging system including an imaging diagnostic apparatus that picks up fluoroscopic image data of a patient, said liquid injector that injects a medical liquid to said patient from whom said fluoroscopic image data is to be picked up, and a data storage unit that stores said fluoroscopic image data together with a patient ID of an individual patient, comprising:
a data input unit that acquires said patient ID;
a liquid injection mechanism that executes injection of said medical liquid;
an injection control unit that controls an action of a liquid injection mechanism based on injection control data;
a data registration unit that registers said injection control data utilized for controlling said action in said data storage unit in association with said fluoroscopic image data;
a data acquisition unit that acquires said injection control data corresponding to said acquired patient ID, from said data storage unit; and
a control setting unit that sets said acquired injection control data in said injection control unit.

37. A liquid injector for use in a fluoroscopic imaging system including an imaging diagnostic apparatus that picks up fluoroscopic image data of a patient, said liquid injector that injects a medical liquid to said patient from whom said fluoroscopic image data is to be picked up, and a data storage unit that stores said fluoroscopic image data together with a patient ID of an individual patient, comprising:
a data input unit that acquires said patient ID;
a liquid injection mechanism that executes injection of said medical liquid;
an injection control unit that controls an action of a liquid injection mechanism based on injection control data;
a history generation unit that generates injection history data including an action history of said liquid injection mechanism corresponding to said injection control data;
a data registration unit that registers said generated injection history data in said data storage unit in association with said fluoroscopic image data;
a data acquisition unit that acquires said injection history data corresponding to said acquired patient ID, from said data storage unit; and
a control setting unit that sets said acquired injection history data as said injection control data, in said injection control unit.

38. The liquid injector according to claim 34,
wherein said data input unit also receives an input of predetermined injection condition data related to an injection condition of said medical liquid;
said data registration unit registers said injection control data together with said patient ID and said injection condition data; and
said data acquisition unit acquires said injection control data corresponding to said patient ID and said injection condition data that have been input.

39. The liquid injector according to claim 35,
wherein said data input unit also receives an input of predetermined injection condition data related to an injection condition of said medical liquid;
said data registration unit registers said injection history data together with said patient ID and said injection condition data; and
said data acquisition unit acquires said injection history data corresponding to said patient ID and said injection condition data that have been input.

40. The liquid injector according to claim 34,
wherein said fluoroscopic imaging system further comprises a liquid syringe including a radio frequency identification (RFID) chip containing at least a product ID of said medical liquid loaded in said syringe;
said data input unit acquires said product ID from said RFID chip in said liquid syringe;
said data registration unit also registers said product ID together with said injection control data;
said data acquisition unit acquires said product ID together with said injection control data;
said liquid injector further comprising: a data confirmation unit that confirms agreement between said product ID acquired with said injection control data and said product ID acquired from said liquid syringe, and
an alert notification unit that outputs a notification of warning in case of disagreement of said product ID.

41. The liquid injector according to claim 35,
wherein said fluoroscopic imaging system further comprises a liquid syringe including a RFID chip containing at least a product ID of said medical liquid loaded in said syringe;
said data input unit acquires said product ID from said RFID chip in said liquid syringe;
said data registration unit also registers said product ID together with said injection history data;
said data acquisition unit acquires said product ID together with said injection history data;
said liquid injector further comprising: a data confirmation unit that confirms agreement between said product ID acquired with said injection history data and said product ID acquired from said liquid syringe, and
an alert notification unit that outputs a notification of warning in case of disagreement of said product ID.

42. The liquid injector according to claim 34,
wherein said fluoroscopic imaging system further comprises a liquid syringe including a RFID chip containing at least an ingredient of said medical liquid loaded in said syringe;
said data input unit acquires said ingredient from said RFID chip in said liquid syringe;
said data registration unit also registers said ingredient together with said injection control data;
said data acquisition unit acquires said ingredient together with said injection control data;
said liquid injector further comprising: a data confirmation unit that confirms agreement between said ingredient acquired with said injection control data and said ingredient acquired from said liquid syringe, and
an alert notification unit that outputs a notification of warning in case of disagreement of said ingredient.

43. The liquid injector according to claim 35,
wherein said fluoroscopic imaging system further comprises a liquid syringe including a RFID chip containing at least an ingredient of said medical liquid loaded in said syringe;
said data input unit acquires said ingredient from said RFID chip in said liquid syringe;
said data registration unit also registers said ingredient together with said injection history data;
said data acquisition unit acquires said ingredient together with said injection history data;
said liquid injector further comprising: a data confirmation unit that confirms agreement between said ingredient acquired with said injection history data and said ingredient acquired from said liquid syringe, and
an alert notification unit that outputs a notification of warning in case of disagreement of said ingredient.

44. The liquid injector according to claim 34,
wherein said fluoroscopic imaging system further comprises a liquid syringe including a RFID chip containing at least a chemical classification of said medical liquid loaded in said syringe;
said data input unit acquires said chemical classification from said RFID chip in said liquid syringe;
said data registration unit also registers said chemical classification together with said injection control data;
said data acquisition unit acquires said chemical classification together with said injection control data;
said liquid injector further comprising: a data confirmation unit that confirms agreement between said chemical classification acquired with said injection control data and said chemical classification acquired from said liquid syringe, and
an alert notification unit that outputs a notification of warning in case of disagreement of said chemical classification.

45. The liquid injector according to claim 35,
wherein said fluoroscopic imaging system further comprises a liquid syringe including a RFID chip containing at least a chemical classification of said medical liquid loaded in said syringe;
said data input unit acquires said chemical classification from said RFID chip in said liquid syringe;
said data registration unit also registers said chemical classification together with said injection history data;
said data acquisition unit acquires said chemical classification together with said injection history data;
said liquid injector further comprising: a data confirmation unit that confirms agreement between said chemical classification acquired with said injection history data and said chemical classification acquired from said liquid syringe, and
an alert notification unit that outputs a notification of warning in case of disagreement of said chemical classification.

46. The liquid injector according to claim 34,
wherein said fluoroscopic imaging system further comprises a patient management medium for each said patient including a RFID chip containing at least said patient ID; and
said data input unit acquires said patient ID from said RFID chip in said patient management medium.

47. The liquid injector according to claim 36,
wherein said fluoroscopic imaging system further comprises an imaging management unit that stores imaging order data in which said patient ID is specified;
said imaging diagnostic apparatus picks up said fluoroscopic image data of said patient according to said imaging order data; and
said data input unit acquires said patient ID from said imaging management unit as a part of said imaging order data.

48. The liquid injector according to claim 47,
wherein said fluoroscopic imaging system further comprises a patient management medium for each said patient including a RFID chip containing at least said patient ID; and
said data input unit acquires said patient ID from said RFID chip in said patient management medium, and also acquires said patient ID from said imaging management unit as at least a part of said imaging order data;
said liquid injector further comprising:
a data confirmation unit that confirms agreement between said patient ID acquired from said RFID chip and said patient ID acquired from said imaging order data, and
an injection restriction unit that inhibits said injection control unit from controlling an action of said liquid injection mechanism until said agreement is confirmed.

49. The liquid injector according to claim 47,
wherein said data input unit acquires said patient ID from said imaging management unit again, once final confirmation right before injection is input; said liquid injector further comprising: a data confirmation unit that confirms agreement between said patient ID previously acquired and said patient ID acquired again, and an injection restriction unit that inhibits said injection control unit from controlling an action of said liquid injection mechanism until said agreement is confirmed.

50. The liquid injector according to claim 48, further comprising an alert notification unit that outputs a predetermined warning in the case where said agreement is not confirmed.

51. The liquid injector according to claim 47,
wherein said fluoroscopic imaging system further comprises a liquid syringe including a RFID chip containing at least a product ID of said medical liquid loaded in said syringe;
said imaging management unit stores said imaging order data containing a product ID of a medical liquid that is inappropriate for injection as an NG ID with respect to each said patient, in association with said patient ID;
said data input unit acquires said product ID from said RFID chip in said liquid syringe, as well as said NG ID from said imaging order data;
said liquid injector further comprising: a data confirmation unit that confirms agreement between said product ID acquired from said liquid syringe and said NG ID acquired from said imaging order data, and
an alert notification unit that outputs a notification of warning in the case where said product ID agrees with said NG ID.

52. The liquid injector according to claim 47,
wherein said fluoroscopic imaging system further comprises a liquid syringe including a RFID chip containing at least an ingredient of said medical liquid loaded in said syringe;
said imaging management unit stores said imaging order data containing an ingredient of a medical liquid that is inappropriate for injection as an NG ingredient with respect to each said patient, in association with said patient ID;
said data input unit acquires said ingredient from said RFID chip in said liquid syringe, as well as said NG ingredient from said imaging order data;
said liquid injector further comprising: a data confirmation unit that confirms agreement between said ingredient acquired from said liquid syringe and said NG ingredient acquired from said imaging order data, and
an alert notification unit that outputs a notification of warning in the case where said ingredient agrees with said NG ingredient.

53. The liquid injector according to claim 47,
wherein said fluoroscopic imaging system further comprises a liquid syringe including a RFID chip containing at least a chemical classification of said medical liquid loaded in said syringe;
said imaging management unit stores said imaging order data containing a chemical classification of a medical liquid that is inappropriate for injection as an NG chemical classification with respect to each said patient, in association with said patient ID;
said data input unit acquires said chemical classification from said RFID chip in said liquid syringe, as well as said NG chemical classification from said imaging order data;
said liquid injector further comprising: a data confirmation unit that confirms agreement between said chemical classification acquired from said liquid syringe and said NG chemical classification acquired from said imaging order data, and
an alert notification unit that outputs a notification of warning in the case where said chemical classification agrees with said NG chemical classification.

54. The liquid injector according to claim 34, further comprising:
a condition listing unit that displays a listing of a plurality of said injection control data acquired based on said patient ID;
wherein said control setting unit sets one of said injection control data displayed in said listing in said injection control unit, in response to a selecting operation.

55. The liquid injector according to claim 34, further comprising:
a condition selection unit that selects, according to a predetermined condition, one of a plurality of said injection control data acquired based on said patient ID;
wherein said control setting unit sets said injection control data thus selected, in said injection control unit.

56. The liquid injector according to claim 55,
wherein said condition selection unit selects a latest one out of said plurality of injection control data acquired.

57. The liquid injector according to claim 34,
wherein said control setting unit newly sets said injection control data, as well as modifies said injection control data, in response to an inputting operation.

58. The liquid injector according to claim 57,
wherein said data input unit also receives an input of predetermined injection condition data related to said injection condition of said medical liquid; and
said control setting unit adjusts said injection control data according to said injection condition data that has been input.

59. The liquid injector according to claim 58,
wherein said fluoroscopic imaging system further comprises a liquid syringe containing said medical liquid, and including a RFID chip containing at least a part of said injection condition data;
said liquid injection mechanism drives said liquid syringe; and
said data input unit acquires at least a part of said injection condition data from said RFID chip.

60. The liquid injector according to claim 58,
wherein said fluoroscopic imaging system further comprises a patient management medium including a RFID chip storing at least a part of said injection condition data and said patient ID; and
said data input unit acquires at least a part of said injection condition data from said RFID chip of said patient management medium.

61. A fluoroscopic imaging system including an imaging diagnostic apparatus that picks up fluoroscopic image data of a patient, a liquid injector that injects a medical liquid to said patient from whom said fluoroscopic image data is to be picked up, and a data storage unit that stores said fluoroscopic image data together with a patient ID of each said patient, comprising:
said liquid injector according to claim 36;
wherein said data storage unit stores said injection control data input from said liquid injector, together with said patient ID.

62. A fluoroscopic imaging system including an imaging diagnostic apparatus that picks up fluoroscopic image data of a patient, a liquid injector that injects a medical liquid to said patient from whom said fluoroscopic image data is to be picked up, a data storage unit that stores said fluoroscopic image data together with a patient ID of each said patient, comprising:
said liquid injector according to claim 37;
wherein said data storage unit stores said injection history data input from said liquid injector, together with said patient ID.

63. A computer program for use with said liquid injector according to claim 34, comprising causing said liquid injector to execute:
a data input process including acquiring a patient ID of an individual patient,
an injection control process including controlling an action of said liquid injection mechanism based on injection control data,
a data registration process including registering said injection control data utilized for controlling said action, together with said patient ID,
a data acquisition process including acquiring said injection control data corresponding to said acquired patient ID from said data registration unit, and
a control setting process including setting said acquired injection control data in said injection control unit.

64. A computer program for use with said liquid injector according to claim 35, comprising causing said liquid injector to execute:
a data input process including acquiring a patient ID of an individual patient,
an injection control process including controlling an action of said liquid injection mechanism based on injection control data,
a history generation process including generating injection history data including an action history of said liquid injection mechanism corresponding to said injection control data,
a data registration process including registering said generated injection history data, together with said patient ID,
a data acquisition process including acquiring said injection history data corresponding to said acquired patient ID input, from said data registration unit, and
a control setting process including setting said acquired injection history data in said injection control unit as said injection control data.

65. A computer program for use with said liquid injector according to claim 36, comprising causing said liquid injector to execute:
a data input process including acquiring a patient ID,
an injection control process including controlling an action of said liquid injection mechanism based on injection control data,
a data registration process including registering said injection control data in said data storage unit in association with said fluoroscopic image data,
a data acquisition process including acquiring said injection control data corresponding to said acquired patient ID from said data storage unit, and
a control setting process including setting said acquired injection control data in said injection control unit.

66. A computer program for use with said liquid injector according to claim 37, comprising causing said liquid injector to execute:
a data input process including acquiring a patient ID,
an injection control process including controlling an action of said liquid injection mechanism based on injection control data
a history generation process including generating injection history data including an action history of said liquid injection mechanism corresponding to said injection control data,
a data registration process including registering said generated injection history data in said data storage unit in association with said fluoroscopic image data,
a data acquisition process including acquiring said injection history data corresponding to said acquired patient ID from said data storage unit, and
a control setting process including setting said acquired injection history data in said injection control unit as said injection control data.
